SEQUENCE LISTING (73 sequences)

SEQ ID NO: 1 - DNA, 21 bp, Artificial sequence
  Synthetic oligonucleotide
gcggctgtagtcactggcagc

SEQ ID NO: 2 - protein, 6 aa, Artificial sequence
  Synthetic peptide
AAVTGS

SEQ ID NO: 3 - DNA, 20 bp, Artificial sequence
  Synthetic oligonucleotide
cccgatgggctggcatgtga

SEQ ID NO: 4 - protein, 4 aa, Artificial sequence
  Synthetic peptide
PDGM

SEQ ID NO: 5 - DNA, 15 bp, Artificial sequence
  Synthetic oligonucleotide
gtctcctccaggtag

SEQ ID NO: 6 - protein, 4 aa, Artificial sequence
  Synthetic peptide
VSSR

SEQ ID NO: 7 - DNA, 24 bp, Artificial sequence
  Synthetic oligonucleotide
ccgcgccgcaagcgggccaagctc

SEQ ID NO: 8 - DNA, 39 bp, Artificial sequence
  Synthetic oligonucleotide
gaggtcatctaccagccgcgccgcaagcgggccaagctc

SEQ ID NO: 9 - DNA, 36 bp, Artificial sequence
  Synthetic oligonucleotide
gaggtcataccagccgcgccgcaacgggccaagctc

SEQ ID NO: 10 - DNA, 40 bp, Artificial sequence
  Synthetic oligonucleotide
gaggtcatctaccagccgcgcccgcaagcgggccaagctc

SEQ ID NO: 11 - DNA, 40 bp, Artificial sequence; identical to SEQ ID NO: 10
  Synthetic oligonucleotide

SEQ ID NO: 12 - DNA, 39 bp, Artificial sequence; identical to SEQ ID NO: 8
  Synthetic oligonucleotide

SEQ ID NO: 13 - DNA, 27 bp, Artificial sequence
  Synthetic oligonucleotide
gagtcatctaccagccgcggcaagctc

SEQ ID NO: 14 - DNA, 39 bp, Artificial sequence; identical to SEQ ID NO: 8
  Synthetic oligonucleotide

SEQ ID NO: 15 - DNA, 37 bp, Artificial sequence
  Synthetic oligonucleotide
gaggtcatctaccagccgcgccaagcgggccaagctc

SEQ ID NO: 16 - DNA, 40 bp, Artificial sequence; identical to SEQ ID NO: 10
  Synthetic oligonucleotide

SEQ ID NO: 17 - DNA, 41 bp, Artificial sequence
  Synthetic oligonucleotide
gaggtcatctaccagccgcgccgcgcaagcgggccaagctc

SEQ ID NO: 18 - DNA, 40 bp, Artificial sequence; identical to SEQ ID NO: 10
  Synthetic oligonucleotide

SEQ ID NO: 19 - DNA, 39 bp, Artificial sequence; identical to SEQ ID NO: 8
  Synthetic oligonucleotide

SEQ ID NO: 20 - DNA, 24 bp, Artificial sequence
  Synthetic oligonucleotide
gaggtccgcaagcgggccaagctc

SEQ ID NO: 21 - DNA, 37 bp, Artificial sequence
  Synthetic oligonucleotide
ccagctcctggccactctgaatccacttcctggctct

SEQ ID NO: 22 - DNA, 33 bp, Artificial sequence
  Synthetic oligonucleotide
ggattggcttgtcctggatattgcatagaaatg

SEQ ID NO: 23 - DNA, 32 bp, Artificial sequence
  Synthetic oligonucleotide
ggatcctgtcggccgggcgcggtgactcacga

SEQ ID NO: 24 - DNA, 37 bp, Artificial sequence
  Synthetic oligonucleotide
gagcaagagatccagaccatcctggccaacatggtga

SEQ ID NO: 25 - DNA, 26 bp, Artificial sequence
  Synthetic oligonucleotide
cagccgcgccgcaagcggccaagctc

SEQ ID NO: 26 - DNA, 25 bp, Artificial sequence
  Synthetic oligonucleotide
cagccgcgccaagcgggccaagctc

SEQ ID NO: 27 - DNA, 28 bp, Artificial sequence
  Synthetic oligonucleotide
cagccgcgcccgcaagcgggccaagctc

SEQ ID NO: 28 - DNA, 22 bp, Artificial sequence
  Synthetic oligonucleotide
agtattctccagaaggtgctaa

SEQ ID NO: 29 - DNA, 26 bp, Artificial sequence
  Synthetic oligonucleotide
tttcctcagtccaggaaggatatttt

SEQ ID NO: 30 - protein, 5 aa, Artificial sequence
  Synthetic peptide
FPRYF

SEQ ID NO: 31 - DNA, 23 bp, Artificial sequence
  Synthetic oligonucleotide
ccattaaagagacctcgagatga

SEQ ID NO: 32 - DNA, 29 bp, Artificial sequence
  Synthetic oligonucleotide
gatcttccagatcctcaagcgatgtttga

SEQ ID NO: 33 - DNA, 22 bp, Artificial sequence
  Synthetic oligonucleotide
ccatcatcaaagagatcgttag

SEQ ID NO: 34 - protein, 5 aa, Artificial sequence
  Synthetic peptide
AIIDR

SEQ ID NO: 35 - DNA, 21 bp, Artificial sequence
  Synthetic oligonucleotide
gcaaatgtctttttcgtggaa

SEQ ID NO: 36 - protein, 7 aa, Artificial sequence
  Synthetic peptide
ANVFFVE

SEQ ID NO: 37 - DNA, 17 bp, Artificial sequence
  Synthetic oligonucleotide
ccccacccgtcaggtga

SEQ ID NO: 38 - protein, 4 aa, Artificial sequence
  Synthetic peptide
AIVR

SEQ ID NO: 39 - DNA, 26 bp, Artificial sequence
  Synthetic oligonucleotide
ttgcggtcattctttgctgtgtatga

SEQ ID NO: 40 - protein, 6 aa, Artificial sequence
  Synthetic peptide
ALLCCV

SEQ ID NO: 41 - DNA, 23 bp, Artificial sequence
  Synthetic oligonucleotide
gccatgcgggcagctggagatga

SEQ ID NO: 42 - protein, 5 aa, Artificial sequence
  Synthetic peptide
LISFR

SEQ ID NO: 43 - DNA, 32 bp, Artificial sequence
  Synthetic oligonucleotide
gagcagatccgggagaccctgaggagagactc

SEQ ID NO: 44 - DNA, 32 bp, Artificial sequence
  Synthetic oligonucleotide
gagcagaccgggagaccctgaggagaggactc

SEQ ID NO: 45 - DNA, 30 bp, Artificial sequence
  Synthetic oligonucleotide
gagcagatccgggagacctgagagagactc

SEQ ID NO: 46 - DNA, 22 bp, Artificial sequence
  Synthetic oligonucleotide
gagcagatccgggagaggactc

SEQ ID NO: 47 - DNA, 31 bp, Artificial sequence
  Synthetic oligonucleotide
gagcagatccgggagaccctggagaggactc

SEQ ID NO: 48 - DNA, 32 bp, Artificial sequence
  Synthetic oligonucleotide
gagcagatccgggagaccctgagagaggactc

SEQ ID NO: 49 - DNA, 33 bp, Artificial sequence
  Synthetic oligonucleotide
gagcagatccgggagaccctgagttccaacttg

SEQ ID NO: 50 - DNA, 32 bp, Artificial sequence
  Synthetic oligonucleotide
gagcagatccgggagaccctgagagggacaac

SEQ ID NO: 51 - DNA, 34 bp, Artificial sequence
  Synthetic oligonucleotide
gagcagatccgggagaccctgagggagaggactc

SEQ ID NO: 52 - DNA, 12 bp, Artificial sequence
  Synthetic oligonucleotide
agcagagaggag

SEQ ID NO: 53 - DNA, 12 bp, Artificial sequence
  Synthetic oligonucleotide
cagatcaggaga

SEQ ID NO: 54 - DNA, 56 bp, Artificial sequence
  Synthetic oligonucleotide
ccaacatggcccgcagagttaatgcagggcttggcacgatacgtgttccagtcagg

SEQ ID NO: 55 - DNA, 29 bp, Artificial sequence
  Synthetic oligonucleotide
ccaatggcacgatacgtgttccagtcagg

SEQ ID NO: 56 - DNA, 54 bp, Artificial sequence
  Synthetic oligonucleotide
ccaacatggcccgcagagtttgcagggcttggcacgatacgtgttccagtcagg

SEQ ID NO: 57 - DNA, 45 bp, Artificial sequence
  Synthetic oligonucleotide
ccaacatggcccgcagagttaatgcagggcttggcacgagtcagg

SEQ ID NO: 58 - DNA, 55 bp, Artificial sequence
  Synthetic oligonucleotide
ccaacatggcccgcagagtaatgcagggcttggcacgatacgtgttccaagcagg

SEQ ID NO: 59 - DNA, 36 bp, Artificial sequence
  Synthetic oligonucleotide
ccttctgctaacatccccgagcactggcactacatc

SEQ ID NO: 60 - DNA, 31 bp, Artificial sequence
  Synthetic oligonucleotide
ccttctgctaacatcccactggcactacatc

SEQ ID NO: 61 - DNA, 25 bp, Artificial sequence
  Synthetic oligonucleotide
ccttctgctaacatccccgagcatc

SEQ ID NO: 62 - DNA, 39 bp, Artificial sequence
  Synthetic oligonucleotide
cctgtccttccaccaatcaaccctcagcggcagaatggc

SEQ ID NO: 63 - DNA, 11 bp, Artificial sequence
  Synthetic oligonucleotide
cgcagaatggc

SEQ ID NO: 64 - DNA, 41 bp, Artificial sequence
  Synthetic oligonucleotide
cctgtccttccaccaaatcaaccctcagccggcagaatggc

SEQ ID NO: 65 - DNA, 12 bp, Artificial sequence
  Synthetic oligonucleotide
caatcagcagaa

SEQ ID NO: 66 - DNA, 12 bp, Artificial sequence
  Synthetic oligonucleotide
aatcaacagaat

SEQ ID NO: 67 - protein, 7 aa, Artificial sequence
  Synthetic peptide
RRKRAKL

SEQ ID NO: 68 - protein, 13 aa, Artificial sequence
  Synthetic peptide
EVIYQPRRKRAKL

SEQ ID NO: 69 - protein, 9 aa, Artificial sequence
  Synthetic peptide
QPRRKRAKL

SEQ ID NO: 70 - protein, 11 aa, Artificial sequence
  Synthetic peptide
EQIRETLRRGL

SEQ ID NO: 71 - protein, 18 aa, Artificial sequence
  Synthetic peptide
PTWPAELMQGLARYVFQS

SEQ ID NO: 72 - protein, 12 aa, Artificial sequence
  Synthetic peptide
PSANIPEHQHYI

SEQ ID NO: 73 - protein, 13 aa, Artificial sequence
  Synthetic peptide
PVLPPINPQRQNG

(19) United States
(12) Patent Application Publication
(10) Pub. No.: US 2018/0353622 A1
(43) Pub. Date: Dec. 13, 2018

(54) GENE DELETION AND RESCUE BY CRISPR-MEDIATED ELIMINATION OF EXON SPLICING ENHANCERS

(71) Applicant: The Board of Regents of the University of Texas System, Austin, TX (US)

(72) Inventors: Lawrence LUM, La Jolla, CA (US); Rubina TULADHAR, Dallas, TX (US); Tae Hyun HWANG, Cleveland, OH (US); Yunku YEU, Cleveland, OH (US); John Tyler PIAZZA, Dallas, TX (US); Quinn BARRETT, Dallas, TX (US)

(21) Appl. No.: 16/003,683

(22) Filed: Jun. 8, 2018

Related U.S. Application Data
(60) Provisional application No. 62/517,723, filed on Jun. 9, 2017.

(51) Int. Cl. A61K 48/00; A61K 38/46; C12N 15/11

(52) U.S. Cl. CPC A61K 48/0066; C12N 2310/20; C12N 15/11; A61K 38/465

(57) ABSTRACT

Disclosed herein are methods for gene inactivation or rescue by cutting genomic DNA at regions of putative exon splicing enhancers with CRISPR-type enzymes.

The present application claims benefit of priority to U.S. Provisional Application Ser. No. 62/517,723, filed Jun. 9, 2017, the entire contents of which are hereby incorporated by reference in their entirety.

This invention was made with government support under Grant No. CA168761, awarded by the National Institutes of Health. The government has certain rights in the invention.

### BACKGROUND OF THE INVENTION

### 1. Field of the Invention

The present invention relates generally to the field of molecular biology. More particularly, it relates to the use CRISPR type enzymes and guide RNAs for the deletion and rescue of genes utilizing exon splicing enhancers.

### 2. Description of Related Art

CRISPR sequences are Clustered Regularly Interspaced Short Palindromic Repeat sequences that are present in bacteria and archaea. CRISPR sequences work together with proteins from the Cas (CRISPR associated) group to form a kind of immune reaction against viral infections (Pennisi, 2013). Recently, it has been found that CRISPR sequences can also work together with a different enzyme, Cpf1 (Zetsche et al., 2015). Genome editing with CRISPR systems frequently results in off-target mutagenesis during this process, including insertions and deletions (indels) formed during the repair process. A number of algorithms have been used to optimize guide RNA sequences for CRISPR associated genome editing, and focus on off-targeting potential, secondary structure of the guide RNA, and whether or not the target sequences encode a critical domain. However, none of these approaches consider the potential impact of altering exon splicing enhancers on the composition of the protein following genome editing. The presence or absence of exon splicing enhancers greatly affects splicing, and could be exploited to achieve gene deletion, or gene rescue if SNPs are present within the gene. Thus, methods to target guide RNAs to exon splicing enhancers may provide significant benefit over current methods.

### SUMMARY OF THE INVENTION

In some embodiments, the present disclosure provides a method for reducing the expression of a target gene in a cell, wherein the target gene comprises a putative exon splicing enhancer sequence, comprising contacting the cell with a gene editing construct and at least one polynucleotide sequence capable of hybridizing to a sequence comprising a putative exon splicing enhancer, or capable of hybridizing to a sequence adjacent to a putative exon splicing enhancer, within the target gene. In some aspects, the putative exon splicing enhancer sequence is selected from the sequences from Table 4. In some aspects, the method further comprises disrupting the putative exon splicing enhancer by inserting or deleting a nucleotide within the sequence during gene editing. In some aspects, said polynucleotide sequence is a vector. In certain aspects, the vector encodes a guide RNA.

In aspects of the embodiments, the guide RNA sequence is about 20 nucleotides in length. In some aspects, the guide RNA sequence is 20 nucleotides in length. In certain aspects, the guide RNA sequence comprises a sequence capable of hybridizing to a sequence adjacent to a putative exon splicing enhancer. In particular aspects, the guide RNA sequence can hybridize to a sequence comprising at least 1, 2, 3, 4, or 5 nucleotides of putative exon splicing enhancer sequence, selected from Table 4, present in the target gene. In some aspects, the guide RNA sequence comprises at least one nucleotide of a protospacer adjacent motif (PAM) sequence. In particular aspects, the at least one nucleotide of the PAM sequence is at the 3′ end of the guide RNA sequence. In specific aspects, the guide RNA comprises a PAM sequence.

In some aspects of the embodiments, the vector is an expression vector. In certain aspects, the expression vector is an adenovirus expression vector, or adeno-associated expression vector. In specific aspects, the vector is an adeno-associated expression vector.

In some aspects, the polynucleotide sequence is an oligonucleotide. In certain aspects, the oligonucleotide is a guide RNA. In particular aspects, the guide RNA sequence is about 20 nucleotides in length. In specific aspects, the guide RNA sequence is 20 nucleotides in length. In some aspects, the guide RNA sequence comprises a sequence capable of hybridizing to a sequence adjacent to a putative exon splicing enhancer sequence. In certain aspects, the putative exon splicing enhancer sequence is selected from the sequences from Table 4. In particular aspects, wherein the guide RNA sequence can hybridize to a sequence comprising at least 1, 2, 3, 4, or 5 nucleotides of putative exon splicing enhancer sequence, selected from Table 4, present in the target gene. In some aspects, the guide RNA sequence comprises at least one nucleotide of a PAM sequence. In specific aspects, the guide RNA comprises a PAM sequence. In certain aspects, the at least one nucleotide of the PAM sequence is at the 3′ end of the guide RNA sequence.

In aspects of the embodiments, the gene editing construct comprises a CRISPR-Cas9, CRISPR-Cas12a, or CRISPR-Cpf1 gene editing construct. In some aspects, the gene editing construct and at least one polynucleotide sequence are co-administered. In other aspects, the gene editing construct is administered prior to the administration of the at least one polynucleotide sequence. In yet other aspects, the gene editing construct is administered subsequent to the administration of the at least one polynucleotide sequence.

In some embodiments, the present disclosure provides a method for skipping expression of a target exon of a gene in a cell, wherein the target exon comprises a putative exon splicing enhancer sequence or is adjacent to a putative exon splicing enhancer sequence, comprising contacting the cell with a gene editing construct and at least one polynucleotide sequence capable of hybridizing to a sequence comprising said putative exon splicing enhancer or hybridizing adjacent to said putative exon splicing enhancer. In some aspects, the putative exon splicing enhancer sequence is selected from the sequences from Table 4. In further aspects, the method further comprises disrupting the putative exon splicing enhancer by inserting or deleting a nucleotide within the sequence during gene editing.

In some aspects, said polynucleotide sequence is a vector. In some aspects of the embodiments the vector is an expression vector. In certain aspects, the expression vector is an adenovirus expression vector, or adeno-associated expression vector. In certain aspects, the vector encodes a guide RNA. In particular aspects, the guide RNA sequence is about 20 nucleotides in length. In specific aspects, the guide RNA sequence is 20 nucleotides in length. In some aspects, the guide RNA sequence comprises a sequence capable of hybridizing to a sequence adjacent to a putative exon splicing enhancer. In certain aspects, the guide RNA sequence can hybridize to a sequence comprising at least 1, 2, 3, 4, or 5 nucleotides of putative exon splicing enhancer sequence, selected from Table 4, present in the target gene. In certain aspects, the guide RNA sequence comprises at least one nucleotide of a PAM sequence. In particular aspects, the guide RNA comprises a PAM motif. In specific aspects, the at least one nucleotide of the PAM motif is at the 3′ end of the guide RNA sequence.

In other aspects, the polynucleotide sequence is an oligonucleotide. In specific aspects, the oligonucleotide is a guide RNA. In certain aspects, the guide RNA sequence is about 20 nucleotides in length. In specific aspects, the guide RNA sequence is 20 nucleotides in length. In some aspects, the guide RNA sequence comprises a sequence capable of hybridizing to a sequence adjacent to a putative exon splicing enhancer. In certain aspects, the guide RNA sequence can hybridize to a sequence comprising at least 1, 2, 3, 4, or 5 nucleotides of putative exon splicing enhancer sequence, selected from Table 4, present in the target gene. In some aspects, the guide RNA sequence comprises at least one nucleotide of a PAM sequence. In certain aspects, the guide RNA comprises a PAM sequence. In specific aspects, the at least one nucleotide of the PAM motif is at the 3′ end of the guide RNA sequence.

In some aspects of the embodiments, the gene editing construct comprises a CRISPR-Cas9, CRISPR-Cas12a, or CRISPR-Cpf1 gene editing construct. In some aspects, the gene editing construct and at least one polynucleotide sequence are co-administered. In other aspects, the gene editing construct is administered prior to the administration of the at least one polynucleotide sequence. In yet other aspects, the gene editing construct is administered subsequent to the administration of the at least one polynucleotide sequence.

In some embodiments, the present disclosure provides a method for treating a subject having a disease caused by a single nucleotide polymorphism in an exon, wherein the exon comprises or is adjacent to an exon splicing enhancer, comprising administering to the subject a therapeutically effective amount of a gene editing construct and at least one polynucleotide sequence capable of hybridizing to a sequence comprising or adjacent to said exon splicing enhancer.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D: Alternative Translation Initiation (ATI) is common in cells with CRISPR/Cas9-introduced INDELs. (FIG. 1A) Depiction of proteins edited by CRISPR/Cas9 and the guide RNAs used in editing. (FIG. 1B) Western blot of edited proteins from HAP1 cells. (FIG. 1C) Reverse transcriptase PCR of the indicated genes and knockouts. (FIG. 1D) Western blot of Lrp6 from 3 different knockouts, with or without PNGase F treatment to cleave N-acetyl glucosamine, preventing translocation of proteins to the cell surface.

FIGS. 2A-C: ATI of Lkb1 introduced by CRISPR/Cas9 editing. (FIG. 2A) Diagram of Lkb1 exons and the guide RNA used for CRISPR/Cas9 editing. (FIG. 2B) Western blots of Lkb1 from selected clones following CRISPR/Cas9 editing. (FIG. 2C) Western blots of Lkb1 from selected clones, with or without Lkb1 siRNA treatment.

FIG. 3: Genome sequencing fails to account for the presence of CRISPR/Cas9 induced proteins. Shown is the amino acid sequence of the region targeted by the sgRNA, as well as the nucleotide sequence with the PAM sequence and sgRNA sequence indicated. The DNA sequence of this region is shown for each of the clones, as well as the length of insertions or deletions.

FIGS. 4A-C: CRISPR/Cas9-mediated disruption of nonsense mediated decay. (FIG. 4A) RT-PCR of the 5′ UTR to exon 4 from Lkb1 from selected clones. Presence of a higher band indicated the possibility of a 131 bp cryptic exon between exons 1 and 2. (FIG. 4B) RT-PCR of the cryptic exon between exons 1 and 2 from selected clones. (FIG. 4C) RT-PCR of WT cells or CRISPR/Cas9 treated clone #8 following treatment with Cyclohexamide to block translation indicating that nonsense mediated decay is reducing the amount of the elongated transcript present in the wild-type.

FIG. 5: CRISPR/Cas9-induced ATI as a consequence of cap-independent translational initiation. Shown at the top are the sgRNA sequence and the sequences found in Clone 48 of the Lkb1 CRISPR/Cas9 knockout. Below, the C-terminal western blot for LKB1 from MIA cells, Clone 48, or Hela cells expressing Lkb1 with the indicated frameshifts, with or without capping enzyme.

FIGS. 6A-C: Induction of foreign protein expression in commercially available CRISPR/Cas9-edited cell lines. (FIG. 6A) Predicted frameshift alterations in CRISPR/Cas9-edited HAP1 clones (Horizon Discovery) relative to the recognition site of antibodies used in the western blots of “B”. (FIG. 6B) Western blot analysis of protein expression in CRISPR/Cas9-edited cell lines using two distinct antibodies. *=novel proteins. (FIG. 6C) RT-PCR analysis and summary of sequencing results of CRISPR/Cas9-edited exons using primers recognizing flanking exons. *=novel amplicons.

FIGS. 7A-D: Frequent exon skipping in CRISPR/Cas9-edited cell lines. (FIG. 7A) Genomic structure of the Suppressor of Fused (SUFU) gene with a location of a single guide RNA sequence targeting Exon 8 (sgRNA 8) used to generate knock-out cell lines in “B”. Exons 8-9 encode a protein epitope recognized by an anti-SUFU antibody used for screening clones. (FIG. 7B) Sequence of sgRNA-targeted genomic region of SUFU Exon 8 from HAP1 cell lines lacking SUFU expression. Indicated below are the locations of putative Exon Splicing Enhancers (ESEs). (FIG. 7C) Western blot analysis of selected HAP1 cell lines with no detectable SUFU expression following transfection with sgRNA 8. (FIG. 7D) RT-PCR analysis of cDNA from putative knock-out clones using primers flanking exon 8, which recognize sequences in exon 6 and 10.

FIGS. 8A-G: Frequent exon skipping in CRISPR/Cas9-edited cell lines. (FIG. 8A) Western blot analysis of RMS13 cell lines transfected with sgRNAs targeting either exon 2, 3, or 8, and which lack SUFU expression. (FIG. 8B) RT-PCR analysis and summary of cDNA sequencing results of Clone 2 and 3 using primers targeting SUFU Exon 1 and 5. (FIG. 8C) RT-PCR analysis and summary of cDNA sequencing results of clones 1 and 4 using a primer pair that generates an amplicon extending from the 5′ UTR to exon 4. (FIG. 8D) RT-PCR and summary of cDNA sequencing results from clones 1 and 4 using a primer pair that generates an amplicon extending from exon 6 to exon 10. (FIG. 8E) Genomic sequencing of the SUFU exon 3 from clones 2 and 3. (FIG. 8F) Genomic sequencing of the SUFU exon 2 region in clone 1, and predicted ESE sequence locations. (FIG. 8G) Genomic sequencing of sgRNA 8 targeted region in clones 1 and 4 and predicted ESE sequence locations.

FIG. 9: Exons skipped from CRISPR associated indels. Shown are the genes targeted, exon targeted, CRISPR induced indels and associated lengths, and exon skipping as a result of the indels.

FIG. 10: Model of CRISPR/Cas9-editing consequences. *=indel location from CRISPR/Cas9-editing. Introduction of indels may lead to alternative translation initiation (ATI), nonsense-mediated decay (NMD), or prevent a protein from being made as a result of the introduction of a premature termination codon (PTC).

FIG. 11: CRISPinatoR: a web-based tool for identifying CRISPR/Cas9 sgRNA sequences which exploit predicted exon splicing enhancer elements found in exonic sequences for gene deletion campaigns. (Left) sgRNA sequences which target ESEs in asymmetric exons are first identified by CRISPinatoR for a given gene of interest. The off-targeting potential and the number of splice variants potentially impacted by any given sgRNA is also incorporated into an overall scoring system that rank orders a list of sgRNAs useful for gene elimination. (Right) The sgRNA design tool Gecko V2, which was used to create a commonly used genome-scale screening library, fails to account for potential interference with exon skipping. sgRNA targeted sequences in the Gecko V2 sgRNA screening library were analyzed by CRISPinatoR and more than 70% of sgRNAs found in the library are anticipated to impact one or more ESEs, potentially making some sgRNAs more effective at promoting gene elimination by targeting asymmetric exons, and others more likely to induce foreign protein expression by targeting symmetric exons.

FIG. 12: CRISPR-skipper: a web based tool for gene rescue by targeting ESEs. SNPs associated with diseases (OMIM and SwissVar databases) found in symmetric exons were identified by CRISPR-skipper. Predicted ESEs that include the SNP or flank the SNP amenable to CRISPR attack are then used to identify sgRNAs with exon skipping potential and low off-targeting scores. Predicted proteins following exon skipping that do not have compromised annotated domain features are identified as viable rescue genes.

FIGS. 13A-B: Comparison of CRISPR-skipper and other CRISPR-associated exon skipping approaches. (FIG. 13A) CRISPR strategies that target either an ESE or splice acceptor (SA)/donor (SD) rely on a single sgRNA, thus reducing the off target potential associated with the use of two sgRNAs in the flanking sgRNA strategy. (FIG. 13B) Comparison of the number of potential sgRNAs within each off-target prediction bracket that could be used for exon skipping for managing inherited diseases (top) and number of SNPs that can be addressed using each strategy (bottom).

FIG. 14: Design of ESE targeting sgRNA.

### DESCRIPTION OF ILLUSTRATIVE EMBODIMENTS

The CRISPR/Cas9 system revolutionized genome editing, but was prone to off target effects. Current algorithms to identify CRISPR guide RNA sequences consider off-target potential, secondary structure of the guide RNA, and whether or not the targeted sequence encodes a critical domain, significantly increasing the efficiency and lowering the off target effects. None of these approaches, however, consider the effects of exon splicing enhancers. Exon splicing enhancers are degenerate hexameric sequences found in nearly every exon which contribute to the splicing of the pre-mRNA transcribed from that gene. The present disclosure provides methods for targeting CRISPR guide RNAs to exon splicing enhancers in order to more efficiently knock down expression of a gene, or to rescue a gene with mutation by skipping that exon. These methods are highly accurate, and yield stable knockouts and gene rescues.

### I. CRISPR Systems

A. CRISPRs

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

B. Cas Nucleases

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E coli) proteins (called CasA-E in E. coli) form a functional complex, Cascade, that processes CRISPR RNA transcripts into spacer-repeat units that Cascade retains. In other prokaryotes, Cash processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. The team demonstrated that they could disable one or both sites while preserving Cas9's ability to locate its target DNA. Jinek et al. (2012) combined tracrRNA and spacer RNA into a “single-guide RNA” molecule that, mixed with Cas9, can find and cut the correct DNA targets and such synthetic guide RNAs are used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Wang et al. (2013) showed that coinjection of Cas9 mRNA and sgRNAs into the germline (zygotes) generated mice with mutations. Delivery of Cas9 DNA sequences also is contemplated.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas systems containing a 1,300 amino acid protein. See also U.S. Patent Publication 2014/0068797, which is incorporated by reference in its entirety.

In some embodiments, the compositions of the disclosure include a small version of a Cas9 from the bacterium Staphylococcus aureus (UniProt Accession No. J7RUA5). The small version of the Cas9 provides advantages over wildtype or full length Cas9. In some embodiments the Cas9 is a spCas9 (AddGene).

C. Cpf1 Nucleases

Clustered Regularly Interspaced Short Palindromic Repeats from Prevotella and Francisella 1 or CRISPR/Cpf1 is a DNA-editing technology which shares some similarities with the CRISPR/Cas9 system. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

Cpf1 appears in many bacterial species. The ultimate Cpf1 endonuclease that was developed into a tool for genome editing was taken from one of the first 16 species known to harbor it.

In embodiments, the Cpf1 is a Cpf1 enzyme from Acidaminococcus, such as species BV3L6, UniProt Accession No. U2UMQ6. In some embodiments, the Cpf1 is a Cpf1 enzyme from Lachnospiraceae, such as species ND2006, UniProt Accession No. A0A182DWE3. In some embodiments, the Cpf1 is codon optimized for expression in mammalian cells. In some embodiments, the Cpf1 is codon optimized for expression in human cells or mouse cells.

The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alpha-helical recognition lobe of Cas9.

Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Database searches suggest the abundance of Cpf1-family proteins in many bacterial species.

Functional Cpf1 does not require a tracrRNA, therefore, only crRNA is required. This benefits genome editing because Cpf1 is not only smaller than Cas9, but also it has a smaller sgRNA molecule (proximately half as many nucleotides as Cas9).

The Cpf1-crRNA complex cleaves target DNA or RNA by identification of a protospacer adjacent motif 5′-YTN-3′ (where “Y” is a pyrimidine and “N” is any nucleobase) or 5′-TTN-3′, in contrast to the G-rich PAM targeted by Cas9. After identification of PAM, Cpf1 introduces a sticky-end-like DNA double-stranded break of 4 or 5 nucleotides overhang.

The CRISPR/Cpf1 system consist of a Cpf1 enzyme and a guide RNA that finds and positions the complex at the correct spot on the double helix to cleave target DNA. CRISPR/Cpf1 systems activity has three stages: Adaptation, formation of crRNAs, and interference. Adaptation is the stage during which Cas1 and Cas2 proteins facilitate the adaptation of small fragments of DNA into the CRISPR array. Formation of crRNAs consists of processing of pre-cr-RNAs producing of mature crRNAs to guide the Cas protein. Interference is when the Cpf1 is bound to a crRNA to form a binary complex to identify and cleave a target DNA sequence.

D. gRNA

As an RNA guided protein, Cas9 requires a short RNA to direct the recognition of DNA targets (Mali et al., 2013a). Though Cas9 preferentially interrogates DNA sequences containing a PAM sequence NGG, it may bind without a protospacer target. However, the Cas9-gRNA complex requires a close match to the gRNA to create a double strand break (Cho et al., 2013; Hsu et al., 2013). CRISPR sequences in bacteria are expressed in multiple RNAs and then processed to create guide strands for RNA (Bikard et al., 2013). Because eukaryotic systems lack some of the proteins required to process CRISPR RNAs the synthetic construct gRNA was created to combine the essential pieces of RNA for Cas9 targeting into a single RNA expressed with the RNA polymerase type III promoter U6 (Mali et al., 2013b,c). Synthetic gRNAs are slightly over 100 bp at the minimum length and contain a portion which is targeted to the 20 protospacer nucleotides immediately preceding the PAM sequence NGG; gRNAs may or may not contain a PAM sequence.

E. Cas9 Versus Cpf1

Cas9 requires two RNA molecules to cut DNA while Cpf1 needs one. The proteins also cut DNA at different places, offering researchers more options when selecting an editing site. Cas9 cuts both strands in a DNA molecule at the same position, leaving behind ‘blunt’ ends. Cpf1 leaves one strand longer than the other, creating ‘sticky’ ends that are easier to work with. Cpf1 appears to be more able to insert new sequences at the cut site, compared to Cas9. Although the CRISPR/Cas9 system can efficiently disable genes, it is challenging to insert genes or generate a knock-in. Cpf1 lacks tracrRNA, utilizes a T-rich PAM and cleaves DNA via a staggered DNA DSB.

In summary, important differences between Cpf1 and Cas9 systems are that Cpf1 recognizes different PAMs, enabling new targeting possibilities, creates 4-5 nt long sticky ends, instead of blunt ends produced by Cas9, enhancing the efficiency of genetic insertions and specificity during NHEJ or HDR, and cuts target DNA further away from PAM, further away from the Cas9 cutting site, enabling new possibilities for cleaving the DNA.

TABLE 1
Differences between Cas9 and Cpf1

| Feature | Cas9 | Cpf1 |
|---|---|---|
| Structure | Two RNA required | One RNA required |
| | (Or 1 fusion transcript | |
| | (crRNA + tracrRNA = gRNA)) | |
| Cutting | Blunt end cuts | Staggered end cuts |
| mechanism | | |
| Cutting site | Proximal to recognition site | Distal from |
| | | recognition site |
| Target sites | G-rich PAM | T-rich PAM |

F. CRISPR-Mediated Gene Editing

The first step in editing a gene using CRISPR/Cpf1 or CRISPR/Cas9 is to identify the genomic target sequence. The genomic target for the gRNAs of the disclosure can be any ˜24 nucleotide DNA sequence, provided that the sequence is unique compared to the rest of the genome.

The next step in editing a gene is to identify all Protospacer Adjacent Motif (PAM) sequences within the genetic region to be targeted. The target sequence must be immediately upstream of a PAM. Once all possible PAM sequences and putative target sites have been identified, the next step is to choose which site is likely to result in the most efficient on-target cleavage. The gRNA targeting sequence needs to match the target sequence, and the gRNA targeting sequence must not match additional sites within the genome. In preferred embodiments, the gRNA targeting sequence has perfect homology to the target with no homology elsewhere in the genome. In some embodiments, a given gRNA targeting sequence will have additional sites throughout the genome where partial homology exists. These sites are called “off-targets” and should be considered when designing a gRNA. In general, off-target sites are not cleaved as efficiently when mismatches occur near the PAM sequence, so gRNAs with no homology or those with mismatches close to the PAM sequence will have the highest specificity. In addition to “off-target activity”, factors that maximize cleavage of the desired target sequence (“on-target activity”) must be considered. It is known to those of skill in the art that two gRNA targeting sequences, each having 100% homology to the target DNA may not result in equivalent cleavage efficiency. In fact, cleavage efficiency may increase or decrease depending upon the specific nucleotides within the selected target sequence. Close examination of predicted on-target and off-target activity of each potential gRNA targeting sequence is necessary to design the best gRNA. Several gRNA design programs have been developed that are capable of locating potential PAM and target sequences and ranking the associated gRNAs based on their predicted on-target and off-target activity (e.g. CRISPRdirect, available at www.crispr.dbcls.jp).

In some embodiments, the gRNA will target a region with an exon splicing enhancer (ESE). When targeting an exon splicing enhancer, the exon splicing enhancer sequence should be located upstream of the PAM sequence, or overlapping the PAM sequence. In particular embodiments, the gRNA will have an ESE positioned between the 15^{th} nucleotide of the gRNA and the first nucleotide of the protospacer adjacent motif sequence. When designing gRNA to target ESE sequences, the web tools CRISPinatoR or CRISPR-skipper may be used. CRISPinator (available at www.crispinator.com) may be used to exploit ESE disruption to eliminate genes. CRISPinatoR works by assigning putative sgRNAs an ESE score prior to an off-target score, and calculates the number of gene splice variants which will be impacted, indicating preferred gRNAs for a gene knockout. Additionally, the CRISPinatoR webtool may be used to design sgRNA for CRISPR-skipper, which exploit ESE disruption to rescue genes, such as those which have a SNP.

The next step is to synthesize and clone desired gRNAs. Targeting oligos can be synthesized, annealed, and inserted into plasmids containing the gRNA scaffold using standard restriction-ligation cloning. However, the exact cloning strategy will depend on the gRNA vector that is chosen. The gRNAs for Cpf1 are notably simpler than the gRNAs for Cas9, and only consist of a single crRNA containing direct repeat scaffold sequence followed by ˜24 nucleotides of guide sequence.

Each gRNA should then be validated in one or more target cell lines. For example, after the Cas9 or Cpf1 and the gRNA are delivered to the cell, the genomic target region may be amplified using PCR and sequenced according to methods known to those of skill in the art.

In some embodiments, gene editing may be performed in vitro or ex vivo. In some embodiments, cells are contacted in vitro or ex vivo with a Cas9 or a Cpf1 and a gRNA that targets a dystrophin splice site. In some embodiments, the cells are contacted with one or more nucleic acids encoding the Cas9 or Cpf1 and the guide RNA. In some embodiments, the one or more nucleic acids are introduced into the cells using, for example, lipofection or electroporation. Gene editing may also be performed in zygotes. In embodiments, zygotes may be injected with one or more nucleic acids encoding Cas9 or Cpf1 and a gRNA that targets a dystrophin splice site. The zygotes may subsequently be injected into a host.

In embodiments, the Cas9 or Cpf1 is provided on a vector. In embodiments, the vector contains a Cas9 derived from S. pyogenes. In embodiments, the vector contains a Cas9 derived from S. aureus. In embodiments, the vector contains a Cpf1 sequence derived from a Lachnospiraceae bacterium. See, for example, Uniprot Accession No. A0A182DWE3. In embodiments, the vector contains a Cpf1 sequence derived from an Acidaminococcus bacterium. See, for example, Uniprot Accession No. U2UMQ6. In some embodiments, the Cas9 or Cpf1 sequence is codon optimized for expression in human cells or mouse cells. In some embodiments, the vector further contains a sequence encoding a fluorescent protein, such as GFP, which allows Cas9 or Cpf1-expressing cells to be sorted using fluorescence activated cell sorting (FACS). In some embodiments, the vector is a viral vector such as an adeno-associated viral vector.

In embodiments, the gRNA is provided on a vector. In some embodiments, the vector is a viral vector such as an adeno-associated viral vector. In embodiments, the Cas9 or Cpf1 and the guide RNA are provided on the same vector. In embodiments, the Cas9 or Cpf1 and the guide RNA are provided on different vectors.

In some embodiments, the cells are additionally contacted with a single-stranded oligonucleotide to effect homology directed repair. In some embodiments, small INDELs restore the protein reading frame of dystrophin (“reframing” strategy). When the reframing strategy is used, the cells may be contacted with a single gRNA. In embodiments, a splice donor or splice acceptor site is disrupted, which results in exon skipping and restoration of the protein reading frame (“exon skipping” strategy). When the exon skipping strategy is used, the cells may be contacted with two or more gRNAs.

Efficiency of in vitro or ex vivo Cas9 or Cpf1-mediated DNA cleavage may be assessed using techniques known to those of skill in the art, such as the T7 E1 assay. Restoration of gene expression may be confirmed using techniques known to those of skill in the art, such as RT-PCR, western blotting, and immunocytochemistry.

In some embodiments, in vitro or ex vivo gene editing is performed in a muscle or satellite cell. In some embodiments, gene editing is performed in iPSC or iCM cells. In embodiments, the iPSC cells are differentiated after gene editing. For example, the iPSC cells may be differentiated into a muscle cell or a satellite cell after editing. In embodiments, the iPSC cells are differentiated into cardiac muscle cells, skeletal muscle cells, or smooth muscle cells. In embodiments, the iPSC cells are differentiated into cardiomyocytes. iPSC cells may be induced to differentiate according to methods known to those of skill in the art.

In some embodiments, contacting the cell with the Cas9 or the Cpf1 and the gRNA restores dystrophin expression. In embodiments, cells which have been edited in vitro or ex vivo, or cells derived therefrom, show levels of dystrophin protein that is comparable to wildtype cells. In embodiments, the edited cells, or cells derived therefrom, express dystrophin at a level that is 50%, 60%, 70%, 80%, 90%, 95% or any percentage in between of wildtype dystrophin expression levels. In embodiments, the cells which have been edited in vitro or ex vivo, or cells derived therefrom, have a mitochondrial number that is comparable to that of wildtype cells. In embodiments the edited cells, or cells derived therefrom, have 50%, 60%, 70%, 80%, 90%, 95% or any percentage in between as many mitochondria as wildtype cells. In embodiments, the edited cells, or cells derived therefrom, show an increase in oxygen consumption rate (OCR) compared to non-edited cells at baseline.

G. RNA Pol III and Pol III Promoters

In eukaryotes, RNA polymerase III (also called Pol III) transcribes DNA to synthesize ribosomal 5S rRNA, tRNA and other small RNAs. The genes transcribed by RNA Pol III fall in the category of “housekeeping” genes whose expression is required in all cell types and most environmental conditions. Therefore, the regulation of Pol III transcription is primarily tied to the regulation of cell growth and the cell cycle, thus requiring fewer regulatory proteins than RNA polymerase II. Under stress conditions however, the protein Maf1 represses Pol III activity.

In the process of transcription (by any polymerase) there are three main stages: (i) initiation, requiring construction of the RNA polymerase complex on the gene's promoter; (ii) elongation, the synthesis of the RNA transcript; and (iii) termination, the finishing of RNA transcription and disassembly of the RNA polymerase complex.

Promoters under the control of RNA Pol III include those for ribosomal 5S rRNA, tRNA and few other small RNAs such as U6 spliceosomal RNA, RNase P and RNase MRP RNA, 7SL RNA (the RNA component of the signal recognition particles), Vault RNAs, Y RNA, SINEs (short interspersed repetitive elements), 7SK RNA, two microRNAs, several small nucleolar RNAs and several few regulatory antisense RNAs

### II. Nucleic Acid Delivery

As discussed above, in certain embodiments, expression cassettes are employed to express a transcription factor product, either for subsequent purification and delivery to a cell/subject, or for use directly in a genetic-based delivery approach. Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy | Banerji et al., 1983; Gilles et al., 1983; |
| Chain | Grosschedl et al., 1985; Atchinson et al., |
| | 1986, 1987; Imler et al., 1987; Weinberger |
| | et al., 1984; Kiledjian et al., 1988; |
| | Porton et al.; 1990 |
| Immunoglobulin Light | Queen and Baltimore, 1983; Picard et al., |
| Chain | 1984 |
| T-Cell Receptor | Luria et al., 1987; Winoto 1989; Redondo |
| | et al; 1990 |
| HLA DQ a and/or DQ β | Sullivan et al., 1987 |
| β-Interferon | Goodbourn et al., 1986; Fujita et al., |
| | 1987; Goodbourn and Maniatis et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase | Jaynes et al., 1988; Horlick et al., 1989; |
| (MCK) | Johnson et al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987a |
| Albumin | Pinkert et al., 1987; Tronche et al., 1989, |
| | 1990 |
| α-Fetoprotein | Godbout et al., 1988; Campere and Tilghman |
| | et al., 1989 |
| t-Globin | Bodine and Ley et al., 1987; Perez-Stable |
| | et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins (GRP94 | |
| and GRP78) | |
| Rat Growth Hormone | Larsen et al., 1986 |
| Human Serum Amyloid | Edbrooke et al., 1989 |
| A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; |
| | Sleigh et al, 1985; Firak et al., 1986; |
| | Herr and Clarke et al., 1986; Imbra and |
| | Karin et al., 1986; Kadesch and Berg, 1986; |
| | Wang et al., 1986; Ondek et al., 1987; |
| | Kuhl et al., 1987; Schaffner et al., 1988 |
| Polyoma | Swartzendruber et al., 1975; Vasseur et |
| | al., 1980; Katinka et al., 1980, 1981; |
| | Tyndell et al., 1981; Dandolo et al., |
| | 1983; de Villiers et al., 1984; Hen et al., |
| | 1986; Satake et al., 1988; Campbell and |
| | Villarreal, 1988 |
| Retroviruses | Kriegler et al., 1982, 1983; Levinson et |
| | al., 1982; Kriegler et al., 1983, 1984a, |
| | b, 1988; Bosze et al., 1986; Miksicek et |
| | al., 1986; Celander and Haseltine, 1987; |
| | Thiesen et al., 1988; Celander et al., |
| | 1988; Choi et al., 1988; Reisman et al., |
| | 1989 |
| Papilloma Virus | Campo et al., 1983; Lusky et al., 1983; |
| | Spandidos and/or Wilkie, 1983; Spalholz |
| | et al., 1985; Lusky et al., 1986; Cripe |
| | et al., 1987; Gloss et al., 1987; |
| | Hirochika et al., 1987; Stephens et al., |
| | 1987 |
| Hepatitis B Virus | Bulla and Siddiqui et al., 1986; Jameel |
| | and Siddiqui, 1986; Shaul and Ben-Levy, |
| | 1987; Spandau et al., 1988; Vannice et |
| | al., 1988 |
| Human | Muesing et al., 1987; Hauber and Cullen |
| Immunodeficiency | et al., 1988; Jakobovits et al., 1988; |
| Virus | Feng et al., 1988; Takebe et al., 1988; |
| | Rosen et al., 1988; Berkhout et al., 1989; |
| | Laspia et al., 1989; Sharp et al., 1989; |
| | Braddock et al., 1989 |
| Cytomegalovirus | Weber et al., 1984; Boshart et al., 1985; |
| (CMV) | Foecking et al., 1986 |
| Gibbon Ape | Holbrook et al., 1987; Quinn et al., 1989 |
| Leukemia Virus | |

TABLE 3
Inducible Elements

| Element | Inducer | References |
|---|---|---|
| MT II | Phorbol Ester (TFA) | Palmiter et al., |
| | Heavy metals | 1982a, b; Haslinger |
| | | et al., 1985; Searle |
| | | et al., 1985; Stuart |
| | | et al., 1985; Imagawa |
| | | et al., 1987, Karin |
| | | et al., 1987; Angel |
| | | et al., 1987b; McNeall |
| | | et al., 1989 |
| MMTV (mouse | Glucocorticoids | Huang et al., 1981; |
| mammary | | Lee et al., 1981; |
| tumor virus) | | Majors and Varmas |
| | | et al., 1983; Chandler |
| | | et al., 1983; Ponta |
| | | et al., 1985; Sakai |
| | | et al., 1988 |
| β-Interferon | poly(rI)x | Tavernier et al., |
| | poly(rc) | 1983 |
| Adenovirus 5 E2 | ElA | Imperiale et al., |
| | | 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large T | Taylor et al., 1989, |
| | Antigen | 1990a, 1990b |
| Proliferin | Phorbol Ester-TPA | Mordacq and Linzer, |
| | | 1989 |
| Tumor Necrosis Factor | PMA | Hensel et al., 1989 |
| Thyroid Stimulating | Thyroid Hormone | Chatterjee et al., |
| Hormone α Gene | | 1989 |

Of particular interest are muscle specific promoters. These include the myosin light chain-2 promoter (Franz et al., 1994; Kelly et al., 1995), the α-actin promoter (Moss et al., 1996), the troponin 1 promoter (Bhaysar et al., 1996); the Na⁺/Ca²⁺ exchanger promoter (Barnes et al., 1997), the dystrophin promoter (Kimura et al., 1997), the α7 integrin promoter (Ziober and Kramer, 1996), the brain natriuretic peptide promoter (LaPointe et al., 1996) and the αB-crystallin/small heat shock protein promoter (Gopal-Srivastava, 1995), α-myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

B. 2A Protease

The invention may utilize the 2A-like self-cleaving domain from the insect virus Thosea asigna (TaV 2A peptide). These 2A-like domains have been shown to function across eukaryotes and cause cleavage of amino acids to occur co-translationally within the 2A-like peptide domain. Therefore, inclusion of TaV 2A peptide allows the expression of multiple proteins from a single mRNA transcript. Importantly, the domain of TaV when tested in eukaryotic systems have shown greater than 99% cleavage activity (Donnelly et al., 2001).

C. Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Baichwal and Sugden, 1986) and adenoviruses (Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNA's for translation.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. disclosed improved methods for culturing 293 cells and propagating adenovirus (Racher et al., 1995). In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10′ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types, however, integration and stable expression require the division of host cells.

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present disclosure. Vectors derived from viruses such as vaccinia virus adeno-associated virus (AAV) and herpesviruses may be employed. They offer several attractive features for various mammalian cells.

In embodiments, the AAV vector is replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof. In some embodiments, a single viral vector is used to deliver a nucleic acid encoding a Cas9 or a Cpf1 and at least one gRNA to a cell. In some embodiments, Cas9 or Cpf1 is provided to a cell using a first viral vector and at least one gRNA is provided to the cell using a second viral vector.

In some embodiments, a single viral vector is used to deliver a nucleic acid encoding Cas9 or Cpf1 and at least one sgRNA to a cell. In some embodiments, Cas9 or Cpf1 is provided to a cell using a first viral vector and at least one gRNA is provided to the cell using a second viral vector. In order to effect expression of sense or antisense gene constructs, the expression construct must be delivered into a cell. The cell may be a muscle cell, a satellite cell, a mesangioblast, a bone marrow derived cell, a stromal cell or a mesenchymal stem cell. In embodiments, the cell is a cardiac muscle cell, a skeletal muscle cell, or a smooth muscle cell. In embodiments, the cell is a cell in the tibialis anterior, quadriceps, soleus, 20 diaphragm or heart. In some embodiments, the cell is an induced pluripotent stem cell (iPSC) or inner cell mass cell (iCM). In further embodiments, the cell is a human iPSC or a human iCM. In some embodiments, human iPSCs or human iCMs of the disclosure may be derived from a cultured stem cell line, an adult stem cell, a placental stem cell, or from another source of adult or embryonic stem cells that does not require the destruction of a human embryo. Delivery to a cell may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al. demonstrated the feasibility of liposome-mediated delivery and expression of foreign DNA in cultured chick embryo, HeLa and hepatoma cells (Wong et al., 1980). Nicolau et al. accomplished successful liposome-mediated gene transfer in rats after intravenous injection (Nicolau et al., 1987). A reagent known as Lipofectamine 2000™ is widely used and commercially available.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). A synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EP 0273085).

### III. Pharmaceutical Compositions and Delivery Methods

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render drugs, proteins or delivery vectors stable and allow for uptake by target cells. Aqueous compositions of the present invention comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

The active compositions of the present invention may include classic pharmaceutical preparations. Administration of these compositions according to the present invention may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### IV. Therapies Section

Diseases and genes which may be amenable to treatment with the methods provided herein include Neoplasia (PTEN; ATM; ATR; EGFR; ERBB2; ERBB3; ERBB4; Notch1; Notch2; Notch3; Notch4; AKT; AKT2; AKT3; HIF; HIF1a; HIF3a; Met; HRG; Bcl2; PPAR alpha; PPAR gamma; WT1 (Wilms Tumor); FGF Receptor Family members (5 members: 1, 2, 3, 4, 5); CDKN2a; APC; RB (retinoblastoma); MEN1; VHL; BRCA1; BRCA2; AR (Androgen Receptor); TSG101; IGF; IGF Receptor; Igf1 (4 variants); Igf2 (3 variants); Igf 1 Receptor; Igf 2 Receptor; Bax; Bcl2; caspases family (9 members: 1, 2, 3, 4, 6, 7, 8, 9, 12); Kras; and Apc), Age related macular degeneration (Abcr; Ccl2; Cc2; cp (ceruloplasmin); Timp3; cathepsinD; Vldlr; Ccr2), Schizophrenia (Neuregulin1 (Nrg1); Erb4 (receptor for Neuregulin); Complexin1 (Cplx1); Tph1 Tryptophan hydroxylase; Tph2 Tryptophan hydroxylase 2; Neurexin 1; GSK3; GSK3a; GSK3b; 5-HTT (Slc6a4); COMT; DRD (Drdla); SLC6A3; DAOA; DTNBP1; Dao (Dao1)), Trinucleotide repeat disorders (HTT (Huntington's Dx); SBMA/SMAX1/AR (Kennedy's Dx); FXN/X25 (Friedrich's Ataxia); ATX3 (Machado-Joseph's Dx); ATXN1 and ATXN2 (spinocerebellar ataxias); DMPK (myotonic dystrophy); Atrophin-1 and Atn1 (DRPLA Dx); CBP (Creb-BP—global instability); VLDLR (Alzheimer's); Atxn7; Atxn10), Fragile X syndrome (FMR2; FXR1; FXR2; mGLUR5), Secretase related disorders (APH-1 (alpha and beta); Presenilin (Psen1); nicastrin (Ncstn); PEN-2), Disorders associated with Nos1, Parp1, Nat1, or Nat2, Prion related disorders (Prp); ALS (SOD1; ALS2; STEX; FUS; TARDBP; VEGF (VEGF-a; VEGF-b; VEGF-c), Drug addiction (Prkce (alcohol); Drd2; Drd4; ABAT (alcohol); GRIA2; Grm5; Grin1; Htr1b; Grin2a; Drd3; Pdyn; Gria1 (alcohol)), autism or autism spectrum disorders (Mecp2; BZRAP1; MDGA2; Sema5A; Neurexin 1; Fragile X (FMR2 (AFF2); FXR1; FXR2; Mglur5)), Alzheimer's Disease (E1; CHIP; UCH; UBB; Tau; LRP; PICALM; Clusterin; PS1; SORL1; CR1; Vldlr; Uba1; Uba3; CHIP28 (Aqp1, Aquaporin 1); Uchl1; Uchl3; APP)), inflammation (IL-10; IL-1 (IL-1a; IL-1b); IL-13; IL-17 (IL-17a (CTLA8); IL-17b; IL-17c; IL-17d; IL-17f); 11-23; Cx3cr1; ptpn22; TNFa; NOD2/CARD15 for IBD; IL-6; IL-12 (IL-12a; IL-12b); CTLA4; Cx3cl1), Parkinson's Disease (x-Synuclein; DJ-1; LRRK2; Parkin; PINK1), blood and coagulation diseases and disorders (Anemia (CDAN1, CDA1, RPS19, DBA, PKLR, PK1, NT5C3, UMPH1, PSN1, RHAG, RH50A, NRAMP2, SPTB, ALAS2, ANH1, ASB, ABCB7, ABC7, ASAT); Bare lymphocyte syndrome (TAPBP, TPSN, TAP2, ABCB3, PSF2, RING11, MHC2TA, C2TA, RFX5, RFXAP, RFX5), Bleeding disorders (TBXA2R, P2RX1, P2X1); Factor H and factor H-like 1 (HF1, CFH, HUS); Factor V and factor VIII (MCFD2); Factor VII deficiency (F7); Factor X deficiency (F10); Factor XI deficiency (F11); Factor XII deficiency (F12, HAF); Factor XIIIA deficiency (F13A1, F13A); Factor XIIIB deficiency (F13B); Fanconi anemia (FANCA, FACA, FA1, FA, FAA, FAAP95, FAAP90, FLJ34064, FANCB, FANCC, FACC, BRCA2, FANCD1, FANCD2, FANCD, FACD, FAD, FANCE, FACE, FANCF, XRCC9, FANCG, BRIP1, BACH1, FANCJ, PHF9, FANCL, FANCM, KIAA1596); Hemophagocytic lymphohistiocytosis disorders (PRF1, HPLH2, UNC13D, MUNC13-4, HPLH3, HLH3, FHL3); Hemophilia A (F8, F8C, HEMA); Hemophilia B (F9, HEMB), Hemorrhagic disorders (PI, ATT, F5); Leukocyde deficiencies and disorders (ITGB2, CD18, LCAMB, LAD, EIF2B1, EIF2BA, EIF2B2, EIF2B3, EIF2B5, LVWM, CACH, CLE, EIF2B4); Sickle cell anemia (HBB); Thalassemia (HBA2, HBB, HBD, LCRB, HBA1)), cell dysregulation and oncology diseases and disorders (B-cell non-Hodgkin lymphoma (BCL7A, BCL7); Leukemia (TAL1, TCL5, SCL, TAL2, FLT3, NBS1, NBS, ZNFN1A1, IK1, LYF1, HOXD4, HOX4B, BCR, CML, PHL, ALL, ARNT, KRAS2, RASK2, GMPS, AF10, ARHGEF12, LARG, KIAA0382, CALM, CLTH, CEBPA, CEBP, CHIC2, BTL, FLT3, KIT, PBT, LPP, NPM1, NUP214, D9S46E, CAN, CAIN, RUNX1, CBFA2, AML1, WHSC1L1, NSD3, FLT3, AF1Q, NPM1, NUMA1, ZNF145, PLZF, PML, MYL, STAT5B, AF10, CALM, CLTH, ARL11, ARLTS1, P2RX7, P2X7, BCR, CML, PHL, ALL, GRAF, NF1, VRNF, WSS, NFNS, PTPN11, PTP2C, SHP2, NS1, BCL2, CCND1, PRAD1, BCL1, TCRA, GATA1, GF1, ERYF1, NFE1, ABL1, NQO1, DIA4, NMOR1, NUP214, D9S46E, CAN, CAIN)), inflammation and immune related diseases and disorders (AIDS (KIR3DL1, NKAT3, NKB1, AMB11, KIR3DS1, IFNG, CXCL12, SDF1); Autoimmune lymphoproliferative syndrome (TNFRSF6, APT1, FAS, CD95, ALPS1A); Combined immunodeficiency, (IL2RG, SCIDX1, SCIDX, IMD4); HIV-1 (CCL5, SCYA5, D17S136E, TCP228), HIV susceptibility or infection (IL10, CSIF, CMKBR2, CCR2, CMKBR5, CCCKR5 (CCR5)); Immunodeficiencies (CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, TACI); Inflammation (IL-10, IL-1 (IL-1a, IL-1b), IL-13, IL-17 (IL-17a (CTLA8), IL-17b, IL-17c, IL-17d, IL-17f), 11-23, Cx3cr1, ptpn22, TNFa, NOD2/CARD15 for IBD, IL-6, IL-12 (IL-12a, IL-12b), CTLA4, Cx3c11); Severe combined immunodeficiencies (SCIDs)(JAK3, JAKL, DCLRE1C, ARTEMIS, SCIDA, RAG1, RAG2, ADA, PTPRC, CD45, LCA, IL7R, CD3D, T3D, IL2RG, SCIDX1, SCIDX, IMD4)), metabolic liver, kidney and protein diseases and disorders (Amyloid neuropathy (TTR, PALB); Amyloidosis (APOA1, APP, AAA, CVAP, AD1, GSN, FGA, LYZ, TTR, PALB); Cirrhosis (KRT18, KRT8, CIRH1A, NAIC, TEX292, KIAA1988); Cystic fibrosis (CFTR, ABCC7, CF, MRP7); Glycogen storage diseases (SLC2A2, GLUT2, G6PC, G6PT, G6PT1, GAA, LAMP2, LAMPB, AGL, GDE, GBE1, GYS2, PYGL, PFKM); Hepatic adenoma, 142330 (TCF1, HNF1A, MODY3), Hepatic failure, early onset, and neurologic disorder (SCOD1, SC01), Hepatic lipase deficiency (LIPC), Hepatoblastoma, cancer and carcinomas (CTNNB1, PDGFRL, PDGRL, PRLTS, AXIN1, AXIN, CTNNB1, TP53, P53, LFS1, IGF2R, MPRI, MET, CASP8, MCH5; Medullary cystic kidney disease (UMOD, HNFJ, FJHN, MCKD2, ADMCKD2); Phenylketonuria (PAH, PKU1, QDPR, DHPR, PTS); Polycystic kidney and hepatic disease (FCYT, PKHD1, ARPKD, PKD1, PKD2, PKD4, PKDTS, PRKCSH, G19P1, PCLD, SEC63)), musculoskeletal diseases and disorders (Becker muscular dystrophy (DMD, BMD, MYF6), Duchenne Muscular Dystrophy (DMD, BMD); Emery-Dreifuss muscular dystrophy (LMNA, LMN1, EMD2, FPLD, CMD1A, HGPS, LGMD1B, LMNA, LMN1, EMD2, FPLD, CMD1A); Facioscapulohumeral muscular dystrophy (FSHMD1A, FSHD1A); Muscular dystrophy (FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDC1C, LGMD2I, TTN, CMD1G, TMD, LGMD2J, POMT1, CAV3, LGMD1C, SEPN1, SELN, RSMD1, PLEC1, PLTN, EBS1); Osteopetrosis (LRP5, BMND1, LRP7, LR3, OPPG, VBCH2, CLCN7, CLC7, OPTA2, OSTM1, GL, TCIRG1, TIRC7, 0C116, OPTB1); Muscular atrophy (VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, SMARD1)), Neurological and neuronal diseases and disorders (ALS (SOD1, ALS2, STEX, FUS, TARDBP, VEGF (VEGF-a, VEGF-b, VEGF-c); Alzheimer disease (APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, AD3); Autism (Mecp2, BZRAP1, MDGA2, Sema5A, Neurexinl, GLO1, MECP2, RTT, PPMX, MRX16, MRX79, NLGN3, NLGN4, KIAA1260, AUTSX2); Fragile X Syndrome (FMR2, FXR1, FXR2, mGLUR5); Huntington's disease and disease like disorders (HD, IT15, PRNP, PRIP, JPH3, JP3, HDL2, TBP, SCA17); Parkinson disease (NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, NDUFV2); Rett syndrome (MECP2, RTT, PPMX, MRX16, MRX79, CDKL5, STK9, MECP2, RTT, PPMX, MRX16, MRX79, x-Synuclein, DJ-1); Schizophrenia (Neuregulin1 (Nrg1), Erb4 (receptor for Neuregulin), Complexin1 (Cplx1), Tph1 Tryptophan hydroxylase, Tph2, Tryptophan hydroxylase 2, Neurexin 1, GSK3, GSK3a, GSK3b, 5-HTT (S1c6a4), COMT, DRD (Drd1a), SLC6A3, DAOA, DTNBP1, Dao (Dao1)); Secretase Related Disorders (APH-1 (alpha and beta), Presenilin (Psen1), nicastrin, (Ncstn), PEN-2, Nos1, Parp1, Nat1, Nat2); Trinucleotide Repeat Disorders (HTT (Huntington's Dx), SBMA/SMAX1/AR (Kennedy's Dx), FXN/X25 (Friedrich's Ataxia), ATX3 (Machado-Joseph's Dx), ATXN1 and ATXN2 (spinocerebellar ataxias), DMPK (myotonic dystrophy), Atrophin-1 and Atn1 (DRPLA Dx), CBP (Creb-BP-global instability), VLDLR (Alzheimer's), Atxn7, Atxn10)), and occular disease and disorders (Age-related macular degeneration (Abcr, Ccl2, Cc2, cp (ceruloplasmin), Timp3, cathepsinD, Vldlr, Ccr2); Cataract (CRYAA, CRYA1, CRYBB2, CRYB2, PITX3, BFSP2, CP49, CP47, CRYAA, CRYA1, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, KRIT1); Corneal clouding and dystrophy (APOA1, TGFBI, CSD2, CDGG1, CSD, BIGH3, CDG2, TACSTD2, TROP2, M1S1, VSX1, RINX, PPCD, PPD, KTCN, COL8A2, FECD, PPCD2, PIP5K3, CFD); Cornea plana congenital (KERA, CNA2); Glaucoma (MYOC, TIGR, GLC1A, JOAG, GPOA, OPTN, GLC1E, FIP2, HYPL, NRP, CYP1B1, GLC3A, OPA1, NTG, NPG, CYP1B1, GLC3A); Leber congenital amaurosis (CRB1, RP12, CRX, CORD2, CRD, RPGRIP1, LCA6, CORDS, RPE65, RP20, AIPL1, LCA4, GUCY2D, GUC2D, LCA1, CORD6, RDH12, LCA3); Macular dystrophy (ELOVL4, ADMD, STGD2, STGD3, RDS, RP7, PRPH2, PRPH, AVMD, AOFMD, VMD2)).

Additionally, signaling pathways and genes may be targeted by the compositions and methods provided herein. Signaling pathways such as PI3K/AKT signaling (PRKCE; ITGAM; ITGA5; IRAK1; PRKAA2; EIF2AK2; PTEN; EIF4E; PRKCZ; GRK6; MAPK1; TSC1; PLK1; AKT2; IKBKB; PIK3CA; CDK8; CDKN1B; NFKB2; BCL2; PIK3CB; PPP2R1A; MAPK8; BCL2L1; MAPK3; TSC2; ITGA1; KRAS; EIF4EBP1; RELA; PRKCD; NOS3; PRKAA1; MAPK9; CDK2; PPP2CA; PIM1; ITGB7; YWHAZ; ILK; TP53; RAF1; IKBKG; RELB; DYRK1A; CDKN1A; ITGB1; MAP2K2; JAK1; AKT1; JAK2; PIK3R1; CHUK; PDPK1; PPP2R5C; CTNNB1; MAP2K1; NFKB1; PAK3; ITGB3; CCND1; GSK3A; FRAP1; SFN; ITGA2; TTK; CSNK1A1; BRAF; GSK3B; AKT3; FOXO1; SGK; HSP90AA1; RPS6KB1), ERK/MAPK signaling (PRKCE; ITGAM; ITGA5; HSPB1; IRAK1; PRKAA2; EIF2AK2; RAC1; RAP1A; TLN1; EIF4E; ELK1; GRK6; MAPK1; RAC2; PLK1; AKT2; PIK3CA; CDK8; CREB1; PRKCI; PTK2; FOS; RPS6KA4; PIK3CB; PPP2R1A; PIK3C3; MAPK8; MAPK3; ITGA1; ETS1; KRAS; MYCN; EIF4EBP1; PPARG; PRKCD; PRKAA1; MAPK9; SRC; CDK2; PPP2CA; PIM1; PIK3C2A; ITGB7; YWHAZ; PPP1CC; KSR1; PXN; RAF1; FYN; DYRK1A; ITGB1; MAP2K2; PAK4; PIK3R1; STAT3; PPP2R5C; MAP2K1; PAK3; ITGB3; ESR1; ITGA2; MYC; TTK; CSNK1A1; CRKL; BRAF; ATF4; PRKCA; SRF; STAT1; SGK), glucocorticoid receptor signaling (RAC1; TAF4B; EP300; SMAD2; TRAF6; PCAF; ELK1; MAPK1; SMAD3; AKT2; IKBKB; NCOR2; UBE2I; PIK3CA; CREB1; FOS; HSPA5; NFKB2; BCL2; MAP3K14; STAT5B; PIK3CB; PIK3C3; MAPK8; BCL2L1; MAPK3; TSC22D3; MAPK10; NRIP1; KRAS; MAPK13; RELA; STAT5A; MAPK9; NOS2A; PBX1; NR3C1; PIK3C2A; CDKN1C; TRAF2; SERPINE1; NCOA3; MAPK14; TNF; RAF1; IKBKG; MAP3K7; CREBBP; CDKN1A; MAP2K2; JAK1; IL8; NCOA2; AKT1; JAK2; PIK3R1; CHUK; STAT3; MAP2K1; NFKB1; TGFBR1; ESR1; SMAD4; CEBPB; JUN; AR; AKT3; CCL2; MMP1; STAT1; IL6; HSP90AA1), axonal guidance signaling (PRKCE; ITGAM; ROCK1; ITGA5; CXCR4; ADAM12; IGF1; RAC1; RAP1A; EIF4E; PRKCZ; NRP1; NTRK2; ARHGEF7; SMO; ROCK2; MAPK1; PGF; RAC2; PTPN11; GNAS; AKT2; PIK3CA; ERBB2; PRKCI; PTK2; CFL1; GNAQ; PIK3CB; CXCL12; PIK3C3; WNT11; PRKD1; GNB2L1; ABL1; MAPK3; ITGA1; KRAS; RHOA; PRKCD; PIK3C2A; ITGB7; GLI2; PXN; VASP; RAF1; FYN; ITGB1; MAP2K2; PAK4; ADAM17; AKT1; PIK3R1; GLI1; WNT5A; ADAM10; MAP2K1; PAK3; ITGB3; CDC42; VEGFA; ITGA2; EPHA8; CRKL; RND1; GSK3B; AKT3; PRKCA), Ephrin receptor signaling (PRKCE; ITGAM; ROCK1; ITGA5; CXCR4; IRAK1; PRKAA2; EIF2AK2; RAC1; RAP1A; GRK6; ROCK2; MAPK1; PGF; RAC2; PTPN11; GNAS; PLK1; AKT2; DOK1; CDK8; CREB1; PTK2; CFL1; GNAQ; MAP3K14; CXCL12; MAPK8; GNB2L1; ABL1; MAPK3; ITGA1; KRAS; RHOA; PRKCD; PRKAA1; MAPK9; SRC; CDK2; PIM1; ITGB7; PXN; RAF1; FYN; DYRK1A; ITGB1; MAP2K2; PAK4; AKT1; JAK2; STAT3; ADAM10; MAP2K1; PAK3; ITGB3; CDC42; VEGFA; ITGA2; EPHA8; TTK; CSNK1A1; CRKL; BRAF; PTPN13; ATF4; AKT3; SGK), actin cytoskeleton signaling (ACTN4; PRKCE; ITGAM; ROCK1; ITGA5; IRAK1; PRKAA2; EIF2AK2; RAC1; INS; ARHGEF7; GRK6; ROCK2; MAPK1; RAC2; PLK1; AKT2; PIK3CA; CDK8; PTK2; CFL1; PIK3CB; MYH9; DIAPH1; PIK3C3; MAPK8; F2R; MAPK3; SLC9A1; ITGA1; KRAS; RHOA; PRKCD; PRKAA1; MAPK9; CDK2; PIM1; PIK3C2A; ITGB7; PPP1CC; PXN; VIL2; RAF1; GSN; DYRK1A; ITGB1; MAP2K2; PAK4; PIP5K1A; PIK3R1; MAP2K1; PAK3; ITGB3; CDC42; APC; ITGA2; TTK; CSNK1A1; CRKL; BRAF; VAV3; SGK), Huntington's disease signaling (PRKCE; IGF1; EP300; RCOR1; PRKCZ; HDAC4; TGM2; MAPK1; CAPNS1; AKT2; EGFR; NCOR2; SP1; CAPN2; PIK3CA; HDAC5; CREB1; PRKCI; HSPA5; REST; GNAQ; PIK3CB; PIK3C3; MAPK8; IGF1R; PRKD1; GNB2L1; BCL2L1; CAPN1; MAPK3; CASP8; HDAC2; HDAC7A; PRKCD; HDAC11; MAPK9; HDAC9; PIK3C2A; HDAC3; TP53; CASP9; CREBBP; AKT1; PIK3R1; PDPK1; CASP1; APAF1; FRAP1; CASP2; JUN; BAX; ATF4; AKT3; PRKCA; CLTC; SGK; HDAC6; CASP3), Apoptosis signaling (PRKCE; ROCK1; BID; IRAK1; PRKAA2; EIF2AK2; BAK1; BIRC4; GRK6; MAPK1; CAPNS1; PLK1; AKT2; IKBKB; CAPN2; CDK8; FAS; NFKB2; BCL2; MAP3K14; MAPK8; BCL2L1; CAPN1; MAPK3; CASP8; KRAS; RELA; PRKCD; PRKAA1; MAPK9; CDK2; PIM1; TP53; TNF; RAF1; IKBKG; RELB; CASP9; DYRK1A; MAP2K2; CHUK; APAF1; MAP2K1; NFKB1; PAK3; LMNA; CASP2; BIRC2; TTK; CSNK1A1; BRAF; BAX; PRKCA; SGK; CASP3; BIRC3; PARP1), B cell receptor signaling (RAC1; PTEN; LYN; ELK1; MAPK1; RAC2; PTPN11; AKT2; IKBKB; PIK3CA; CREB1; SYK; NFKB2; CAMK2A; MAP3K14; PIK3CB; PIK3C3; MAPK8; BCL2L1; ABL1; MAPK3; ETS1; KRAS; MAPK13; RELA; PTPN6; MAPK9; EGR1; PIK3C2A; BTK; MAPK14; RAF1; IKBKG; RELB; MAP3K7; MAP2K2; AKT1; PIK3R1; CHUK; MAP2K1; NFKB1; CDC42; GSK3A; FRAP1; BCL6; BCL10; JUN; GSK3B; ATF4; AKT3; VAV3; RPS6KB1), leukocyte extravasation signaling (ACTN4; CD44; PRKCE; ITGAM; ROCK1; CXCR4; CYBA; RAC1; RAP1A; PRKCZ; ROCK2; RAC2; PTPN11; MMP14; PIK3CA; PRKCI; PTK2; PIK3CB; CXCL12; PIK3C3; MAPK8; PRKD1; ABL1; MAPK10; CYBB; MAPK13; RHOA; PRKCD; MAPK9; SRC; PIK3C2A; BTK; MAPK14; NOX1; PXN; VIL2; VASP; ITGB1; MAP2K2; CTNND1; PIK3R1; CTNNB1; CLDN1; CDC42; F11R; ITK; CRKL; VAV3; CTTN; PRKCA; MMP1; MMP9), integrin signaling (ACTN4; ITGAM; ROCK1; ITGA5; RAC1; PTEN; RAP1A; TLN1; ARHGEF7; MAPK1; RAC2; CAPNS1; AKT2; CAPN2; PIK3CA; PTK2; PIK3CB; PIK3C3; MAPK8; CAV1; CAPN1; ABL1; MAPK3; ITGA1; KRAS; RHOA; SRC; PIK3C2A; ITGB7; PPP1CC; ILK; PXN; VASP; RAF1; FYN; ITGB1; MAP2K2; PAK4; AKT1; PIK3R1; TNK2; MAP2K1; PAK3; ITGB3; CDC42; RND3; ITGA2; CRKL; BRAF; GSK3B; AKT3), acute phase response signaling (IRAK1; SOD2; MYD88; TRAF6; ELK1; MAPK1; PTPN11; AKT2; IKBKB; PIK3CA; FOS; NFKB2; MAP3K14; PIK3CB; MAPK8; RIPK1; MAPK3; IL6ST; KRAS; MAPK13; IL6R; RELA; SOCS1; MAPK9; FTL; NR3C1; TRAF2; SERPINE1; MAPK14; TNF; RAF1; PDK1; IKBKG; RELB; MAP3K7; MAP2K2; AKT1; JAK2; PIK3R1; CHUK; STAT3; MAP2K1; NFKB1; FRAP1; CEBPB; JUN; AKT3; IL1R1; IL6), PTEN signaling (ITGAM; ITGA5; RAC1; PTEN; PRKCZ; BCL2L11; MAPK1; RAC2; AKT2; EGFR; IKBKB; CBL; PIK3CA; CDKN1B; PTK2; NFKB2; BCL2; PIK3CB; BCL2L1; MAPK3; ITGA1; KRAS; ITGB7; ILK; PDGFRB; INSR; RAF1; IKBKG; CASP9; CDKN1A; ITGB1; MAP2K2; AKT1; PIK3R1; CHUK; PDGFRA; PDPK1; MAP2K1; NFKB1; ITGB3; CDC42; CCND1; GSK3A; ITGA2; GSK3B; AKT3; FOXO1; CASP3; RPS6KB1), P53 signaling (PTEN; EP300; BBC3; PCAF; FASN; BRCA1; GADD45A; BIRC5; AKT2; PIK3CA; CHEK1; TP53INP1; BCL2; PIK3CB; PIK3C3; MAPK8; THBS1; ATR; BCL2L1; E2F1; PMAIP1; CHEK2; TNFRSF10B; TP73; RB1; HDAC9; CDK2; PIK3C2A; MAPK14; TP53; LRDD; CDKN1A; HIPK2; AKT1; PIK3R1; RRM2B; APAF1; CTNNB1; SIRT1; CCND1; PRKDC; ATM; SFN; CDKN2A; JUN; SNAI2; GSK3B; BAX; AKT3), Aryl Hydrocarbon Receptor Signaling (HSPB1; EP300; FASN; TGM2; RXRA; MAPK1; NQO1; NCOR2; SP1; ARNT; CDKN1B; FOS; CHEK1; SMARCA4; NFKB2; MAPK8; ALDH1A1; ATR; E2F1; MAPK3; NRIP1; CHEK2; RELA; TP73; GSTP1; RB1; SRC; CDK2; AHR; NFE2L2; NCOA3; TP53; TNF; CDKN1A; NCOA2; APAF1; NFKB1; CCND1; ATM; ESR1; CDKN2A; MYC; JUN; ESR2; BAX; IL6; CYP1B1; HSP90AA1), Xenobiotic Metabolism Signaling (PRKCE; EP300; PRKCZ; RXRA; MAPK1; NQO1; NCOR2; PIK3CA; ARNT; PRKCI; NFKB2; CAMK2A; PIK3CB; PPP2R1A; PIK3C3; MAPK8; PRKD1; ALDH1A1; MAPK3; NRIP1; KRAS; MAPK13; PRKCD; GSTP1; MAPK9; NOS2A; ABCB1; AHR; PPP2CA; FTL; NFE2L2; PIK3C2A; PPARGC1A; MAPK14; TNF; RAF1; CREBBP; MAP2K2; PIK3R1; PPP2R5C; MAP2K1; NFKB1; KEAP1; PRKCA; EIF2AK3; IL6; CYP1B1; HSP90AA1), SAPK/JNK Signaling (PRKCE; IRAK1; PRKAA2; EIF2AK2; RAC1; ELK1; GRK6; MAPK1; GADD45A; RAC2; PLK1; AKT2; PIK3CA; FADD; CDK8; PIK3CB; PIK3C3; MAPK8; RIPK1; GNB2L1; IRS1; MAPK3; MAPK10; DAXX; KRAS; PRKCD; PRKAA1; MAPK9; CDK2; PIM1; PIK3C2A; TRAF2; TP53; LCK; MAP3K7; DYRK1A; MAP2K2; PIK3R1; MAP2K1; PAK3; CDC42; JUN; TTK; CSNK1A1; CRKL; BRAF; SGK), PPAr/RXR Signaling (PRKAA2; EP300; INS; SMAD2; TRAF6; PPARA; FASN; RXRA; MAPK1; SMAD3; GNAS; IKBKB; NCOR2; ABCA1; GNAQ; NFKB2; MAP3K14; STAT5B; MAPK8; IRS1; MAPK3; KRAS; RELA; PRKAA1; PPARGC1A; NCOA3; MAPK14; INSR; RAF1; IKBKG; RELB; MAP3K7; CREBBP; MAP2K2; JAK2; CHUK; MAP2K1; NFKB1; TGFBR1; SMAD4; JUN; IL1R1; PRKCA; IL6; HSP90AA1; ADIPOQ), NF-KB Signaling (IRAK1; EIF2AK2; EP300; INS; MYD88; PRKCZ; TRAF6; TBK1; AKT2; EGFR; IKBKB; PIK3CA; BTRC; NFKB2; MAP3K14; PIK3CB; PIK3C3; MAPK8; RIPK1; HDAC2; KRAS; RELA; PIK3C2A; TRAF2; TLR4; PDGFRB; TNF; INSR; LCK; IKBKG; RELB; MAP3K7; CREBBP; AKT1; PIK3R1; CHUK; PDGFRA; NFKB1; TLR2; BCL10; GSK3B; AKT3; TNFAIP3; IL1R1), Neuregulin Signaling (ERBB4; PRKCE; ITGAM; ITGA5; PTEN; PRKCZ; ELK1; MAPK1; PTPN11; AKT2; EGFR; ERBB2; PRKCI; CDKN1B; STAT5B; PRKD1; MAPK3; ITGA1; KRAS; PRKCD; STAT5A; SRC; ITGB7; RAF1; ITGB1; MAP2K2; ADAM17; AKT1; PIK3R1; PDPK1; MAP2K1; ITGB3; EREG; FRAP1; PSEN1; ITGA2; MYC; NRG1; CRKL; AKT3; PRKCA; HSP90AA1; RPS6KB1), Wnt & Beta catenin signaling (CD44; EP300; LRP6; DVL3; CSNK1E; GJA1; SMO; AKT2; PIN1; CDH1; BTRC; GNAQ; MARK2; PPP2R1A; WNT11; SRC; DKK1; PPP2CA; SOX6; SFRP2; ILK; LEF1; SOX9; TP53; MAP3K7; CREBBP; TCF7L2; AKT1; PPP2R5C; WNT5A; LRP5; CTNNB1; TGFBR1; CCND1; GSK3A; DVL1; APC; CDKN2A; MYC; CSNK1A1; GSK3B; AKT3; SOX2), Insulin Receptor signaling (PTEN; INS; EIF4E; PTPN1; PRKCZ; MAPK1; TSC1; PTPN11; AKT2; CBL; PIK3CA; PRKCI; PIK3CB; PIK3C3; MAPK8; IRS1; MAPK3; TSC2; KRAS; EIF4EBP1; SLC2A4; PIK3C2A; PPP1CC; INSR; RAF1; FYN; MAP2K2; JAK1; AKT1; JAK2; PIK3R1; PDPK1; MAP2K1; GSK3A; FRAP1; CRKL; GSK3B; AKT3; FOXO1; SGK; RPS6KB1), IL-6 Signaling (HSPB1; TRAF6; MAPKAPK2; ELK1; MAPK1; PTPN11; IKBKB; FOS; NFKB2; MAP3K14; MAPK8; MAPK3; MAPK10; IL6ST; KRAS; MAPK13; IL6R; RELA; SOCS1; MAPK9; ABCB1; TRAF2; MAPK14; TNF; RAF1; IKBKG; RELB; MAP3K7; MAP2K2; IL8; JAK2; CHUK; STAT3; MAP2K1; NFKB1; CEBPB; JUN; IL1R1; SRF; IL6), Hepatic Cholestasis (PRKCE; IRAK1; INS; MYD88; PRKCZ; TRAF6; PPARA; RXRA; IKBKB; PRKCI; NFKB2; MAP3K14; MAPK8; PRKD1; MAPK10; RELA; PRKCD; MAPK9; ABCB1; TRAF2; TLR4; TNF; INSR; IKBKG; RELB; MAP3K7; IL8; CHUK; NR1H2; TJP2; NFKB1; ESR1; SREBF1; FGFR4; JUN; IL1R1; PRKCA; IL6), IGF-1 Signaling (IGF1; PRKCZ; ELK1; MAPK1; PTPN11; NEDD4; AKT2; PIK3CA; PRKCI; PTK2; FOS; PIK3CB; PIK3C3; MAPK8; IGF1R; IRS1; MAPK3; IGFBP7; KRAS; PIK3C2A; YWHAZ; PXN; RAF1; CASP9; MAP2K2; AKT1; PIK3R1; PDPK1; MAP2K1; IGFBP2; SFN; JUN; CYR61; AKT3; FOXO1; SRF; CTGF; RPS6KB1), NRF2-mediated oxidative stress response (PRKCE; EP300; SOD2; PRKCZ; MAPK1; SQSTM1; NQO1; PIK3CA; PRKCI; FOS; PIK3CB; PIK3C3; MAPK8; PRKD1; MAPK3; KRAS; PRKCD; GSTP1; MAPK9; FTL; NFE2L2; PIK3C2A; MAPK14; RAF1; MAP3K7; CREBBP; MAP2K2; AKT1; PIK3R1; MAP2K1; PPIB; JUN; KEAP1; GSK3B; ATF4; PRKCA; EIF2AK3; HSP90AA1), hepatic fibrosis/hepatic stellate cell activation (EDN1; IGF1; KDR; FLT1; SMAD2; FGFR1; MET; PGF; SMAD3; EGFR; FAS; CSF1; NFKB2; BCL2; MYH9; IGF1R; IL6R; RELA; TLR4; PDGFRB; TNF; RELB; IL8; PDGFRA; NFKB1; TGFBR1; SMAD4; VEGFA; BAX; IL1R1; CCL2; HGF; MMP1; STAT1; IL6; CTGF; MMP9), PPAR Signaling (EP300; INS; TRAF6; PPARA; RXRA; MAPK1; IKBKB; NCOR2; FOS; NFKB2; MAP3K14; STAT5B; MAPK3; NRIP1; KRAS; PPARG; RELA; STAT5A; TRAF2; PPARGC1A; PDGFRB; TNF; INSR; RAF1; IKBKG; RELB; MAP3K7; CREBBP; MAP2K2; CHUK; PDGFRA; MAP2K1; NFKB1; JUN; IL1R1; HSP90AA1), Fc Epsilon RI Signaling (PRKCE; RAC1; PRKCZ; LYN; MAPK1; RAC2; PTPN11; AKT2; PIK3CA; SYK; PRKCI; PIK3CB; PIK3C3; MAPK8; PRKD1; MAPK3; MAPK10; KRAS; MAPK13; PRKCD; MAPK9; PIK3C2A; BTK; MAPK14; TNF; RAF1; FYN; MAP2K2; AKT1; PIK3R1; PDPK1; MAP2K1; AKT3; VAV3; PRKCA), G-protein coupled receptor signaling (PRKCE; RAP1A; RGS16; MAPK1; GNAS; AKT2; IKBKB; PIK3CA; CREB1; GNAQ; NFKB2; CAMK2A; PIK3CB; PIK3C3; MAPK3; KRAS; RELA; SRC; PIK3C2A; RAF1; IKBKG; RELB; FYN; MAP2K2; AKT1; PIK3R1; CHUK; PDPK1; STAT3; MAP2K1; NFKB1; BRAF; ATF4; AKT3; PRKCA), inositol phosphate metabolism (PRKCE; IRAK1; PRKAA2; EIF2AK2; PTEN; GRK6; MAPK1; PLK1; AKT2; PIK3CA; CDK8; PIK3CB; PIK3C3; MAPK8; MAPK3; PRKCD; PRKAA1; MAPK9; CDK2; PIM1; PIK3C2A; DYRK1A; MAP2K2; PIP5K1A; PIK3R1; MAP2K1; PAK3; ATM; TTK; CSNK1A1; BRAF; SGK), PDGF signaling (EIF2AK2; ELK1; ABL2; MAPK1; PIK3CA; FOS; PIK3CB; PIK3C3; MAPK8; CAV1; ABL1; MAPK3; KRAS; SRC; PIK3C2A; PDGFRB; RAF1; MAP2K2; JAK1; JAK2; PIK3R1; PDGFRA; STAT3; SPHK1; MAP2K1; MYC; JUN; CRKL; PRKCA; SRF; STAT1; SPHK2), VEGF signaling (ACTN4; ROCK1; KDR; FLT1; ROCK2; MAPK1; PGF; AKT2; PIK3CA; ARNT; PTK2; BCL2; PIK3CB; PIK3C3; BCL2L1; MAPK3; KRAS; HIF1A; NOS3; PIK3C2A; PXN; RAF1; MAP2K2; ELAVL1; AKT1; PIK3R1; MAP2K1; SFN; VEGFA; AKT3; FOXO1; PRKCA), Natural killer cell signaling (PRKCE; RAC1; PRKCZ; MAPK1; RAC2; PTPN11; KIR2DL3; AKT2; PIK3CA; SYK; PRKCI; PIK3CB; PIK3C3; PRKD1; MAPK3; KRAS; PRKCD; PTPN6; PIK3C2A; LCK; RAF1; FYN; MAP2K2; PAK4; AKT1; PIK3R1; MAP2K1; PAK3; AKT3; VAV3; PRKCA), Cell Cycle: G1/S checkpoint regulation (HDAC4; SMAD3; SUV39H1; HDAC5; CDKN1B; BTRC; ATR; ABL1; E2F1; HDAC2; HDAC7A; RB1; HDAC11; HDAC9; CDK2; E2F2; HDAC3; TP53; CDKN1A; CCND1; E2F4; ATM; RBL2; SMAD4; CDKN2A; MYC; NRG1; GSK3B; RBL1; HDAC6), T cell receptor signaling (RAC1; ELK1; MAPK1; IKBKB; CBL; PIK3CA; FOS; NFKB2; PIK3CB; PIK3C3; MAPK8; MAPK3; KRAS; RELA; PIK3C2A; BTK; LCK; RAF1; IKBKG; RELB; FYN; MAP2K2; PIK3R1; CHUK; MAP2K1; NFKB1; ITK; BCL10; JUN; VAV3), Death receptor signaling (CRADD; HSPB1; BID; BIRC4; TBK1; IKBKB; FADD; FAS; NFKB2; BCL2; MAP3K14; MAPK8; RIPK1; CASP8; DAXX; TNFRSF10B; RELA; TRAF2; TNF; IKBKG; RELB; CASP9; CHUK; APAF1; NFKB1; CASP2; BIRC2; CASP3; BIRC3), FGF signaling (RAC1; FGFR1; MET; MAPKAPK2; MAPK1; PTPN11; AKT2; PIK3CA; CREB1; PIK3CB; PIK3C3; MAPK8; MAPK3; MAPK13; PTPN6; PIK3C2A; MAPK14; RAF1; AKT1; PIK3R1; STAT3; MAP2K1; FGFR4; CRKL; ATF4; AKT3; PRKCA; HGF), GM-CSF signaling (LYN; ELK1; MAPK1; PTPN11; AKT2; PIK3CA; CAMK2A; STAT5B; PIK3CB; PIK3C3; GNB2L1; BCL2L1; MAPK3; ETS1; KRAS; RUNX1; PIM1; PIK3C2A; RAF1; MAP2K2; AKT1; JAK2; PIK3R1; STAT3; MAP2K1; CCND1; AKT3; STAT1), Amyotrophic lateral sclerosis (ALS) signaling (BID; IGF1; RAC1; BIRC4; PGF; CAPNS1; CAPN2; PIK3CA; BCL2; PIK3CB; PIK3C3; BCL2L1; CAPN1; PIK3C2A; TP53; CASP9; PIK3R1; RAB5A; CASP1; APAF1; VEGFA; BIRC2; BAX; AKT3; CASP3; BIRC3), JAK/Stat Signaling (PTPN1; MAPK1; PTPN11; AKT2; PIK3CA; STAT5B; PIK3CB; PIK3C3; MAPK3; KRAS; SOCS1; STAT5A; PTPN6; PIK3C2A; RAF1; CDKN1A; MAP2K2; JAK1; AKT1; JAK2; PIK3R1; STAT3; MAP2K1; FRAP1; AKT3; STAT1), Nicotinate and Nicotinamide metabolism (PRKCE; IRAK1; PRKAA2; EIF2AK2; GRK6; MAPK1; PLK1; AKT2; CDK8; MAPK8; MAPK3; PRKCD; PRKAA1; PBEF1; MAPK9; CDK2; PIM1; DYRK1A; MAP2K2; MAP2K1; PAK3; NT5E; TTK; CSNK1A1; BRAF; SGK), Chemokine Signaling (CXCR4; ROCK2; MAPK1; PTK2; FOS; CFL1; GNAQ; CAMK2A; CXCL12; MAPK8; MAPK3; KRAS; MAPK13; RHOA; CCR3; SRC; PPP1CC; MAPK14; NOX1; RAF1; MAP2K2; MAP2K1; JUN; CCL2; PRKCA), IL-2 Signaling (ELK1; MAPK1; PTPN11; AKT2; PIK3CA; SYK; FOS; STAT5B; PIK3CB; PIK3C3; MAPK8; MAPK3; KRAS; SOCS1; STAT5A; PIK3C2A; LCK; RAF1; MAP2K2; JAK1; AKT1; PIK3R1; MAP2K1; JUN; AKT3), Synaptic long term depression (PRKCE; IGF1; PRKCZ; PRDX6; LYN; MAPK1; GNAS; PRKCI; GNAQ; PPP2R1A; IGF1R; PRKD1; MAPK3; KRAS; GRN; PRKCD; NOS3; NOS2A; PPP2CA; YWHAZ; RAF1; MAP2K2; PPP2R5C; MAP2K1; PRKCA), estrogen receptor signaling (TAF4B; EP300; CARM1; PCAF; MAPK1; NCOR2; SMARCA4; MAPK3; NRIP1; KRAS; SRC; NR3C1; HDAC3; PPARGC1A; RBM9; NCOA3; RAF1; CREBBP; MAP2K2; NCOA2; MAP2K1; PRKDC; ESR1; ESR2), Protein ubiquitination pathway (TRAF6; SMURF1; BIRC4; BRCA1; UCHL1; NEDD4; CBL; UBE2I; BTRC; HSPA5; USP7; USP10; FBXW7; USP9X; STUB1; USP22; B2M; BIRC2; PARK2; USPS; USP1; VHL; HSP90AA1; BIRC3), IL-10 Signaling (TRAF6; CCR1; ELK1; IKBKB; SP1; FOS; NFKB2; MAP3K14; MAPK8; MAPK13; RELA; MAPK14; TNF; IKBKG; RELB; MAP3K7; JAK1; CHUK; STAT3; NFKB1; JUN; IL1R1; IL6), VDR/RXR Activation (PRKCE; EP300; PRKCZ; RXRA; GADD45A; HES1; NCOR2; SP1; PRKCI; CDKN1B; PRKD1; PRKCD; RUNX2; KLF4; YY1; NCOA3; CDKN1A; NCOA2; SPP1; LRP5; CEBPB; FOXO1; PRKCA), TGF-beta Signaling (EP300; SMAD2; SMURF1; MAPK1; SMAD3; SMAD1; FOS; MAPK8; MAPK3; KRAS; MAPK9; RUNX2; SERPINE1; RAF1; MAP3K7; CREBBP; MAP2K2; MAP2K1; TGFBR1; SMAD4; JUN; SMAD5), Toll-like Receptor signaling (IRAK1; EIF2AK2; MYD88; TRAF6; PPARA; ELK1; IKBKB; FOS; NFKB2; MAP3K14; MAPK8; MAPK13; RELA; TLR4; MAPK14; IKBKG; RELB; MAP3K7; CHUK; NFKB1; TLR2; JUN), p38 MAPK Signaling (HSPB1; IRAK1; TRAF6; MAPKAPK2; ELK1; FADD; FAS; CREB1; DDIT3; RPS6KA4; DAXX; MAPK13; TRAF2; MAPK14; TNF; MAP3K7; TGFBR1; MYC; ATF4; IL1R1; SRF; STAT1), Neurotrophin/TRK Signaling (NTRK2; MAPK1; PTPN11; PIK3CA; CREB1; FOS; PIK3CB; PIK3C3; MAPK8; MAPK3; KRAS; PIK3C2A; RAF1; MAP2K2; AKT1; PIK3R1; PDPK1; MAP2K1; CDC42; JUN; ATF4), FXR/RXR Activation (INS; PPARA; FASN; RXRA; AKT2; SDC1; MAPK8; APOB; MAPK10; PPARG; MTTP; MAPK9; PPARGC1A; TNF; CREBBP; AKT1; SREBF1; FGFR4; AKT3; FOXO1), Synaptic Long Term Potentiation (PRKCE; RAP1A; EP300; PRKCZ; MAPK1; CREB1; PRKCI; GNAQ; CAMK2A; PRKD1; MAPK3; KRAS; PRKCD; PPP1CC; RAF1; CREBBP; MAP2K2; MAP2K1; ATF4; PRKCA), Calcium Signaling (RAP1A; EP300; HDAC4; MAPK1; HDAC5; CREB1; CAMK2A; MYH9; MAPK3; HDAC2; HDAC7A; HDAC11; HDAC9; HDAC3; CREBBP; CALR; CAMKK2; ATF4; HDAC6), EGF Signaling (ELK1; MAPK1; EGFR; PIK3CA; FOS; PIK3CB; PIK3C3; MAPK8; MAPK3; PIK3C2A; RAF1; JAK1; PIK3R1; STAT3; MAP2K1; JUN; PRKCA; SRF; STAT1), Hypoxia Signaling in the cardiovascular system (EDN1; PTEN; EP300; NQO1; UBE2I; CREB1; ARNT; HIF1A; SLC2A4; NOS3; TP53; LDHA; AKT1; ATM; VEGFA; JUN; ATF4; VHL; HSP90AA1), LPS/IL-1 mediated inhibition of RXR function (IRAK1; MYD88; TRAF6; PPARA; RXRA; ABCA1; MAPK8; ALDH1A1; GSTP1; MAPK9; ABCB1; TRAF2; TLR4; TNF; MAP3K7; NR1H2; SREBF1; JUN; IL1R1), LXR/RXR Activation (FASN; RXRA; NCOR2; ABCA1; NFKB2; IRF3; RELA; NOS2A; TLR4; TNF; RELB; LDLR; NR1H2; NFKB1; SREBF1; IL1R1; CCL2; IL6; MMP9), Amyloid Processing (PRKCE; CSNK1E; MAPK1; CAPNS1; AKT2; CAPN2; CAPN1; MAPK3; MAPK13; MAPT; MAPK14; AKT1; PSEN1; CSNK1A1; GSK3B; AKT3; APP), IL-4 Signaling (AKT2; PIK3CA; PIK3CB; PIK3C3; IRS1; KRAS; SOCS1; PTPN6; NR3C1; PIK3C2A; JAK1; AKT1; JAK2; PIK3R1; FRAP1; AKT3; RPS6KB1), Cell Cycle: G2/M DNA damage checkpoint regulation (EP300; PCAF; BRCA1; GADD45A; PLK1; BTRC; CHEK1; ATR; CHEK2; YWHAZ; TP53; CDKN1A; PRKDC; ATM; SFN; CDKN2A), Nitric Oxide Signaling in the cardiovascular system (KDR; FLT1; PGF; AKT2; PIK3CA; PIK3CB; PIK3C3; CAV1; PRKCD; NOS3; PIK3C2A; AKT1; PIK3R1; VEGFA; AKT3; HSP90AA1), Purine Metabolism (NME2; SMARCA4; MYH9; RRM2; ADAR; EIF2AK4; PKM2; ENTPD1; RADS1; RRM2B; TJP2; RAD51C; NT5E; POLD1; NME1), cAMP-mediated signaling (RAP1A; MAPK1; GNAS; CREB1; CAMK2A; MAPK3; SRC; RAF1; MAP2K2; STAT3; MAP2K1; BRAF; ATF4), Mitochondrial dysfunction (SOD2; MAPK8; CASP8; MAPK10; MAPK9; CASP9; PARK7; PSEN1; PARK2; APP; CASP3), Notch Signaling (HES1; JAG1; NUMB; NOTCH4; ADAM17; NOTCH2; PSEN1; NOTCH3; NOTCH1; DLL4), Endoplasmic Reticulum stress pathway (HSPA5; MAPK8; XBP1; TRAF2; ATF6; CASP9; ATF4; EIF2AK3; CASP3), Pyrimidine Metabolism (NME2; AICDA; RRM2; EIF2AK4; ENTPD1; RRM2B; NT5E; POLD1; NME1), Parkinson's Signaling (UCHL1; MAPK8; MAPK13; MAPK14; CASP9; PARK7; PARK2; CASP3), Cardiac & Beta adrenergic signaling (GNAS; GNAQ; PPP2R1A; GNB2L1; PPP2CA; PPP1CC; PPP2R5C), Glycolysis/Gluconeogenesis (HK2; GCK; GPI; ALDH1A1; PKM2; LDHA; HK1), Interferon Signaling (IRF1; SOCS1; JAK1; JAK2; IFITM1; STAT1; IFIT3), Sonic Hedgehog signaling (ARRB2; SMO; GLI2; DYRK1A; GLI1; GSK3B; DYRK1B), Glycerophospholipid metabolism (PLD1; GRN; GPAM; YWHAZ; SPHK1; SPHK2), Phospholipid degradation (PRDX6; PLD1; GRN; YWHAZ; SPHK1; SPHK2), Tryptophan Metabolism (SIAH2; PRMT5; NEDD4; ALDH1A1; CYP1B1; SIAH1), Lysine Degradation (SUV39H1; EHMT2; NSD1; SETD7; PPP2R5C), Nucleotide excisions repair pathway (ERCC5; ERCC4; XPA; XPC; ERCC1), Starch and Sucrose metabolism (UCHL1; HK2; GCK; GPI; HK1), Aminosugars Metabolism (NQO1; HK2; GCK; HK1), Arachidonic Acid metabolism (PRDX6; GRN; YWHAZ; CYP1B1), Circadian Rhythm signaling (CSNK1E; CREB1; ATF4; NR1D1), Coagulation System (BDKRB1; F2R; SERPINE1; F3), Dopamine receptor signaling (PPP2R1A; PPP2CA; PPP1CC; PPP2R5C), Glutathione Metabolism (IDH2; GSTP1; ANPEP; IDH1), Glycerolipid Metabolism (ALDH1A1; GPAM; SPHK1; SPHK2), Linoleic acid metabolism (PRDX6; GRN; YWHAZ; CYP1B1), Methionine Metabolism (DNMT1; DNMT3B; AHCY; DNMT3A), Pyruvate Metabolism (GLO1; ALDH1A1; PKM2; LDHA), Arginine and Proline metabolism (ALDH1A1; NOS3; NOS2A), Eicosanoid Signaling (PRDX6; GRN; YWHAZ), Fructose and Mannose metabolism (HK2; GCK; HK1), Galactose Metabolism (HK2; GCK; HK1), Stilbene, Coumarine and Lignin biosynthesis (PRDX6; PRDX1; TYR), Antigen Presentation Pathway (CALR; B2M), Biosynthesis of Steroids (NQO1; DHCR7), Butanoate Metabolism (ALDH1A1; NLGN1), Citrate Cycle (IDH2; IDH1), Fatty Acid Metabolism (ALDH1A1; CYP1B1), Glycerophospholipid metabolism (PRDX6; CHKA), Histidine Metabolism (PRMT5; ALDH1A1), Inositol Metabolism (ERO1L; APEX1), Metabolism of xenobiotics by Cytocrhome p450 (GSTP1; CYP1B1), Methane Metabolism (PRDX6; PRDX1), Phenylalanine metabolism (PRDX6; PRDX1), Propanoate Metabolism (ALDH1A1; LDHA), Selenoamino Acid metabolism (PRMT5; AHCY), Sphingolipid Metabolism (SPHK1; SPHK2), Aminophosphonate metabolism (PRMT5), Androgen and Estrogen metabolism (PRMT5), Ascorbate and Aldarate metabolism (ALDH1A1), Bile Acid Biosynthesis (ALDH1A1), Cysteine Metabolism (LDHA), Fatty Acid Biosynthesis (FASN), Glutamate Receptor signaling (GNB2L1), NRF2-mediated oxidative stress response (PRDX1), Pentose Phosphate pathway (GPI), Pentose and Glucuronate interconversions (UCHL1), Retinol Metabolism (ALDH1A1), Riboflavin Metabolism (TYR), Tyrosine Metabolism (PRMT5, TYR), Ubiquinone Biosynthesis (PRMT5), Valine, Leucine and Isoleucine degradation (ALDH1A1), Glycine, Serine and Threonine metabolism (CHKA), Lysine Degradation (ALDH1A1), Pain/Taste (TRPM5; TRPA1), Pain (TRPM7; TRPC5; TRPC6; TRPC1; Cnr1; cnr2; Grk2; Trpa1; Pomc; Cgrp; Crf; Pka; Era; Nr2b; TRPM5; Prkaca; Prkacb; Prkar1a; Prkar2a), Mitochondrial Function (AIF; CytC; SMAC (Diablo); Aifm-1; Aifm-2), Developmental neurology (BMP-4; Chordin (Chrd); Noggin (Nog); WNT (Wnt2; Wnt2b; Wnt3a; Wnt4; Wnt5a; Wnt6; Wnt7b; Wnt8b; Wnt9a; Wnt9b; Wnt10a; Wnt10b; Wnt16); beta-catenin; Dkk-1; Frizzled related proteins; Otx-2; Gbx2; FGF-8; Reelin; Dab1; unc-86 (Pou4f1 or Brn3a); Numb; Reln).

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Western Blot Analysis.

Cell lysates were generated with PBS/1% NP40 buffer supplemented with protease inhibitor cocktail (Sigma). Antibodies were purchased from the following sources: Cell Signaling Technology (Lkb1 C-term, Lkb1 N-term, Axin1, Sirt1, Rictor, Sufu, Lrp6, Bclw), Millipore (Ctnnb1), Bethyl Laboratories (Sirt1, Top1, Vps35, Rictor), Abcam (Ppm1a, Tle3, Bap1), Santa Cruz Biotechnology (Lkb1 N-term, Lrp6, Pten, Tbk1), and Genetex (Vps35).

Transfection of sgRNAs.

1×10⁶ Mia pancreatic cells were seeded per 6 well and co-transfected with 0.35 μg sgRNA pCas-Guide plasmid and 0.15 μg puromycin expression plasmid using effectene transfection reagent (Qiagen). 24 hours after transfection, cells were trypisinized and plated in 150 mm culture dishes in various dilutions for clonal selection.

Clonal Isolation of CRISPR-Edited Cells.

Cells in 150 mm culture plates were treated with 0.5 ug/ml of puromycin in order to select for the cells with expression of pCas-Guide and puromycin plasmid. Puromycin selection was maintained for 10 days after which single colonies were isolated and grown in a 96 well plate. Cells from single colonies were passaged multiple times until sufficient cells were available for analyzing genomic DNA, RNA and protein.

Genomic DNA Extraction and Genomic Sequencing.

Genomic DNA was extracted from the CRISPR-edited cells using Genomic DNA Mini kit (Bioland Scientific) according to manufacturer's instructions and used as template for PCR amplification. PCR primers encompassing the CRIPSR-targeted region were designed. PCR was performed with GoTaq Green Master Mix (Promega M7122) with the PCR following condition: 98° □C for 2 mins (Initial denaturation), 25 cycles of 98° C. for 30 secs, 56° C. for 30 secs, 72° C. for 30 secs (denaturation, annealing, extension) and final 70° C. for 5 minutes (final extension). Gel electrophoresis in a 1% agarose gel was performed and the PCR products were purified from gel using QIA Quick PCR Purification Kit (Qiagen) and cloned into pCR-TOPO plasmid using TOPO TA cloning kit for Subcloning (ThermoFisher Scientific). pCR-TOPO plasmids containing genomic DNA sequences were transformed into TOP10 competent cells and individual colonies were selected and sequenced at UTSW Sequencing Core.

RNA Extraction and Analysis.

RNA extraction was performed using RNeasy Mini Kit (Qiagen) according to manufacturer's instructions. cDNA synthesis was performed on 1 μg of RNA using ProtoScript First Strand cDNA Synthesis Kit (Promega). Primers recognizing distal exons within the targeted gene were designed and used to amplify the cDNA sequences isolated from the CRISPR-edited cells. PCR products were electrophoresed in 1% agarose gel and the gel bands were isolated using QIA Quick PCR Purification Kit (Qiagen), cloned into pCR-TOPO plasmids using TOPO TA cloning kit for subcloning (ThermoFisher Scientific) and sequenced at UTSW Sequencing Core.

### Example 2—Alternative Translation Initiation and Exon Skipping as a Consequence of CRISPR/Cas9 Editing

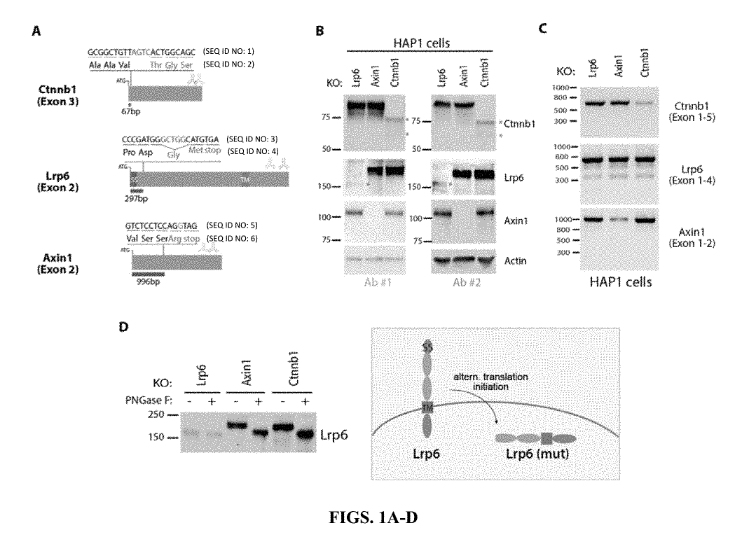

Current algorithms to identify CRISPR guide RNA sequences consider off-target potential, secondary structure of the guide RNA, and whether or not the targeted sequence encodes a critical domain, however none of these approaches consider the effects of alternative translation initiation or exon splicing enhancers. This can result in unintended consequences, such as alternative translation initiation. FIG. 1A shows several proteins which were edited by CRISPR/Cas9, and the guide RNAs used. Following CRISPR/Cas9 treatment of HAP1 cells, expression of these genes was examined by western blot and RT-PCR (FIGS. 1B-1C). As seen in FIG. 1B, the Ctnnb1 and Lrp6 proteins show truncated versions present in the Western blots, indicating that the frameshift may have allowed for a second translation initiation site to be used, as the RT-PCR results do not indicate a change in size, just mRNA expression. Further, each of the knockout strains was treated with PNGase F to determine whether the size and intensity of the Lrp6 band was a result of the knockout, or whether it was a result of N-acetyl-glucosamine cleavage. As indicated in FIG. 1D, the size of the Lrp6 knockout and PNGase F treated bands is similar, indicating the protein likely was not transported out of the cell, and was a consequence of alternative translation initiation.

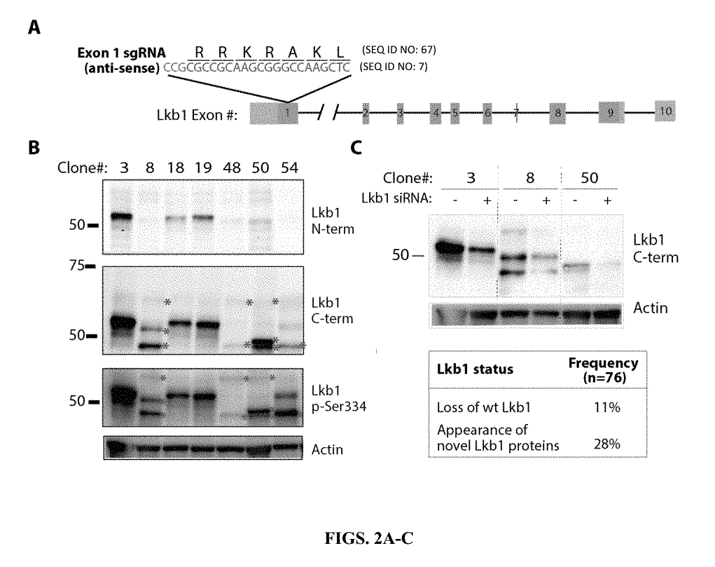

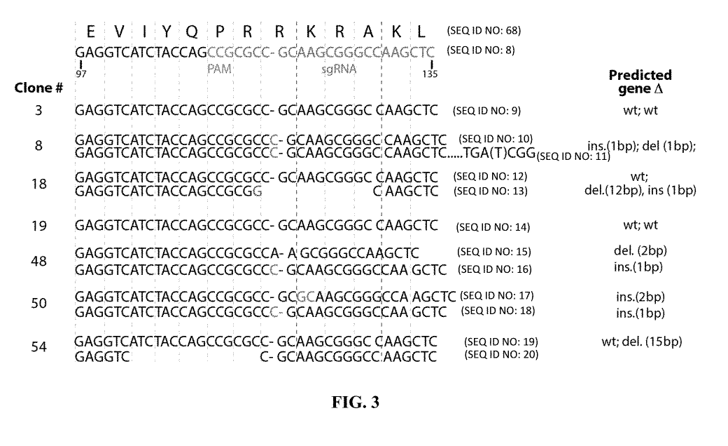

Another demonstration of alternative translation initiation as a consequence of CRISPR/Cas9 editing is shown in FIGS. 2A-C. Briefly, Lkb1 is edited in Mia pancreatic cells by CRISPR/Cas9 using the sgRNA shown in FIG. 2A. 76 clones were generated, of which 11% had loss of Lkb1, while 28% actually showed novel Lkb1 proteins as detected by Western blot. FIG. 2B shows Western blots of several clones with varying degrees of Lkb1 expression, and varying fragment sizes. For example, Clones 8, 48, 50, and 54 display two or more bands at sizes which do not correspond to Lkb1. To confirm that these were in fact Lkb1 products which were created by alternative translation initiation, each of these clones was treated with Lkb1 siRNA (FIG. 2C). Expression of Lkb1 products was significantly decreased in clones 3, 8 and 50 following siRNA treatment, indicating that these products are the result of alternative translation initiation. Further, the Lkb1 sequence of each of these clones was sequenced to determine whether the target region was modified in such a way to induce the formation of these proteins (FIG. 3). Interestingly, the sequencing fails to account for the presence of these proteins.

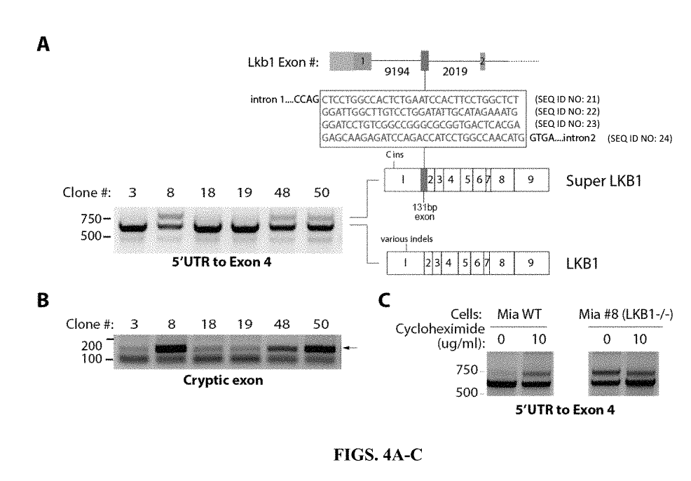

It was further hypothesized that CRISPR/Cas9 editing may have an effect on nonsense mediated decay (NMD), as several clones exhibited a fragment larger than Lkb1. It was noted that a cryptic exon exists in the intron between exons 1 and 2 (FIG. 4A). RT-PCR of the Lkb1 transcript was performed on clones 3, 8, 18, 19, 48, and 50 and a fragment larger than than WT was found in clones 8, 48, and 50 (FIG. 4A). RT-PCR was then performed amplifying the fragment between exons 1 and 2, and clones 8, 48, and 50 displayed a strong band above normal, while 3, 18, and 19 displayed faint bands. It was thus hypothesized that wild-type cells process this larger band by nonsense mediated decay. To test this, wild-type cells or clone number 8 were either untreated or treated with cyclohexamide to block translation, and then the Lkb1 transcript was amplified to determine the presence or absence of the cryptic exon. As shown in FIG. 4C, cyclohexamide treatment allowed for the buildup of the transcript with the cryptic exon in wild-type cells, while clone number 8 always showed the larger band, indicating that the CRISPR/Cas9 disrupted the nonsense mediated decay of this transcript.

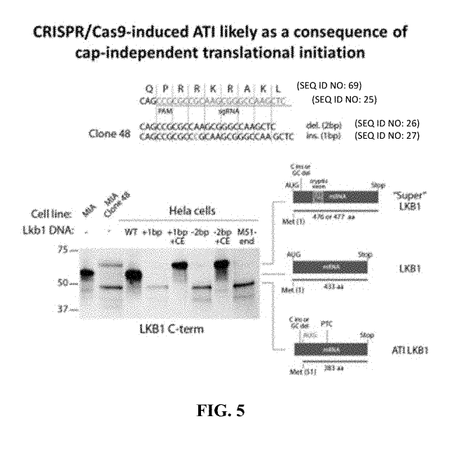

Next, it was investigated whether alternative translation initiation was independent of traditional cap-dependent translation. Wild-type Mia cells and clone 8 were investigated alongside Hela cells expressing either wild-type Lkb1, or Lkb1 with +1, or −2 frameshifts, with or without capping enzyme. As can be seen in FIG. 5, Lkb1 expressing cells with frameshifts without capping enzyme express truncated Lkb1. However, once supplemented with capping enzyme, both frameshifts reveal a product which includes the cryptic exon. These results indicate that alternative translation initiation resulting in the truncated products is independent of the cap, while capped transcripts produce “super” Lkb1 which includes the cryptic exon.

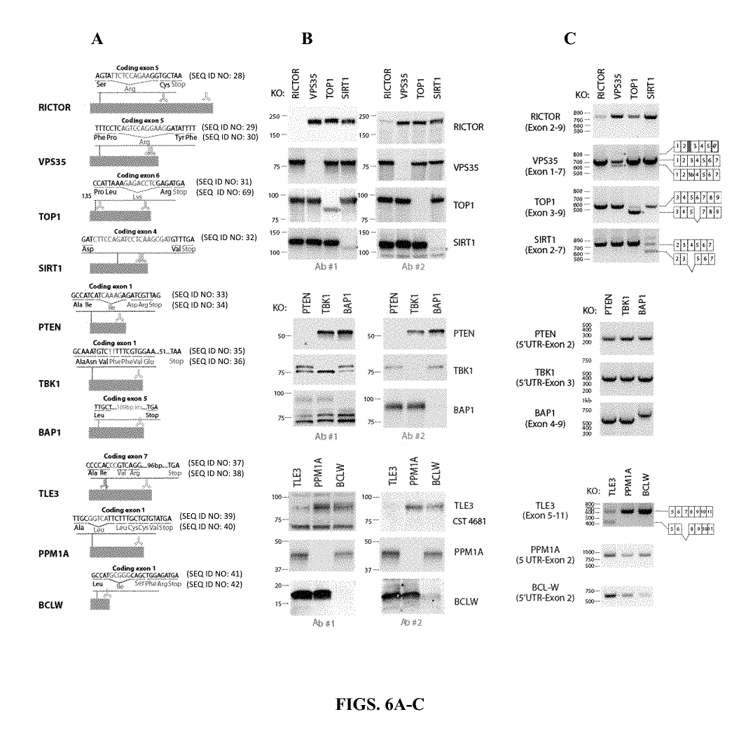

Examination of a number of commercial CRISPR/Cas9-edited cell lines show the induction of foreign proteins as well. FIG. 6 shows the predicted frameshift alteration in a number of CRISPR/Cas9 edited HAP1 clones (Horizon Discover) relative to the the recognition site of their antibodies. Each of the proteins indicated in FIG. 6 was tested for expression by Western blot analysis in CRISPR/Cas9-edited cell lines using two distinct antibodies (FIG. 6, center). TOP1 and SIRT1 edited cells show novel proteins (indicated by an asterisk) and RT-PCR fragments (FIG. 6, center and right columns). Additionally, VP35 showed novel bands in RT-PCR, including a larger band indicating the presence of a cryptic exon (FIG. 6, right column).

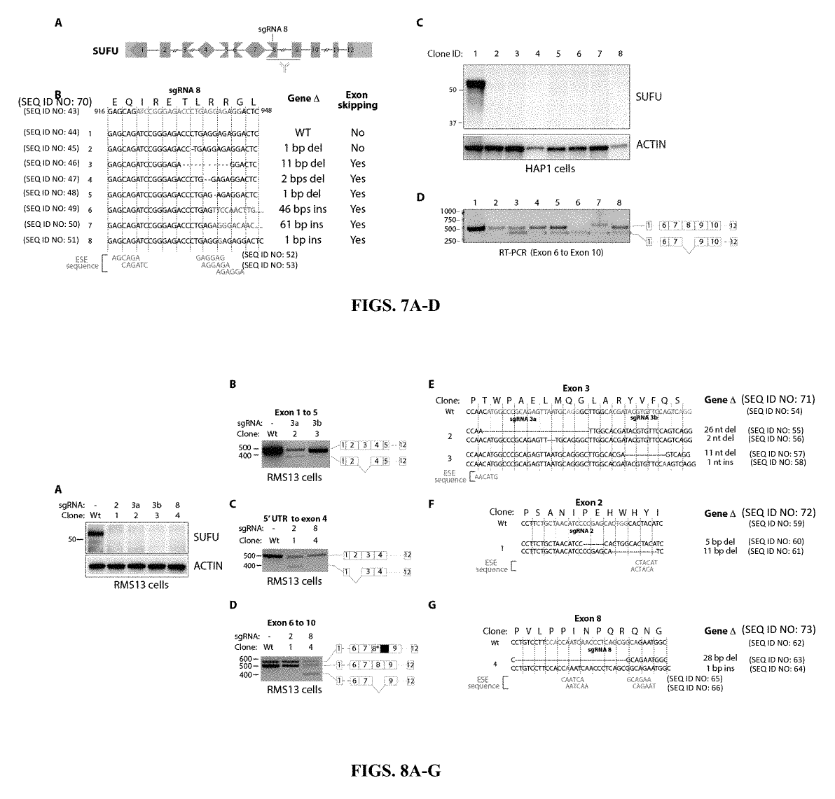

To better understand the mechanism by which these aberrant products were produced, a CRISPR/Cas9 knockout of the Suppressor of Fused (SUFU) was generated using a single guide RNA targeted to exon 8 (FIG. 7A). Exons 8-9 encode a protein epitope recognized by an anti-SUFU antibody used for screening clones. From the knockout, eight distinct clones which lacked SUFU expression, as viewed by Western blot, were generated (FIG. 7C). These clones were then sequenced, and a variety of mutations were found in the SUFU gene following CRISPR/Cas9 editing with sgRNA8 (FIG. 7B). To understand the consequences of the CRISPR/Cas9 editing, RT-PCR was performed with primers directed to exon 6 and 10 (FIG. 7D). Interestingly, clones 3, 4, 5, 6, 7 and 8 each showed a distinct band beneath where the WT SUFU band was located, which corresponds to a product missing exon 8. Each of these clones had an indel in a putative exon splicing enhancer sequence, indicating that the compromised exon splicing enhancers may account for the exon skipping.

To confirm the idea that indels in exon splicing enhancer sequences account for exon skipping, RMS13 cells were transfected with sgRNAs directed to exons 2, 3 and 8 of SUFU, and the SUFU gene was edited by CRISPR/Cas9. Clones were selected in which SUFU expression was knocked out, as viewed by Western Blot (FIG. 8A). To understand the transcripts being generated, RT-PCR was performed using primers to either exons 1 and 5 (FIG. 8B), the 5′ UTR and exon 4 (FIG. 8C), or exons 6 and 10 (FIG. 8D). As can be seen in FIG. 8B, knockout using sgRNA3a results in 2 bands, while knockout with sgRNA 3b results in in a single band. Sequencing of the SUFU gene from each knockout indicates that there is a disruption to an ESE sequence in Clone 2 (sgRNA 3a) (FIG. 8E), which has caused the third exon to be skipped (FIG. 8B). Similarly, when clones 1 and 4 were analyzed by RT-PCR (FIG. 8C) a second band is present in clone 1. As shown in FIG. 8F, a deletion as a result of editing with sgRNA 2 has disrupted the ESE sequence, and thus exon 2 is skipped. Examination of exons 6 through 10 by RT-PCR reveal that CRISPR/Cas9 editing with sgRNA 8 results in an extra band, in which exon 8 has been skipped (FIG. 8D). Sequence analysis shows that a 28 bp deletion in one strand affects several ESE sequences (FIG. 8G), and is likely the cause of the exon skipping, though the other strand does include a single bp insertion which may affect one of the ESEs. Further, examination of a variety of indels induced by CRISPR confirms that the majority of CRISPR-induced indels which hit ESEs result in exon skipping (FIG. 9). In fact, 11 of the 14 indels which compromise the ESE sequence result in exon skipping, whereas only 1 of the 10 indels which does not compromise the ESE sequence results in exon skipping. Therefore ESE targeting by CRISPR induced indels is a viable way to knock out specific exons, and genes.

### Example 3—Targeting Exon Splicing Enhancers with CRISPR

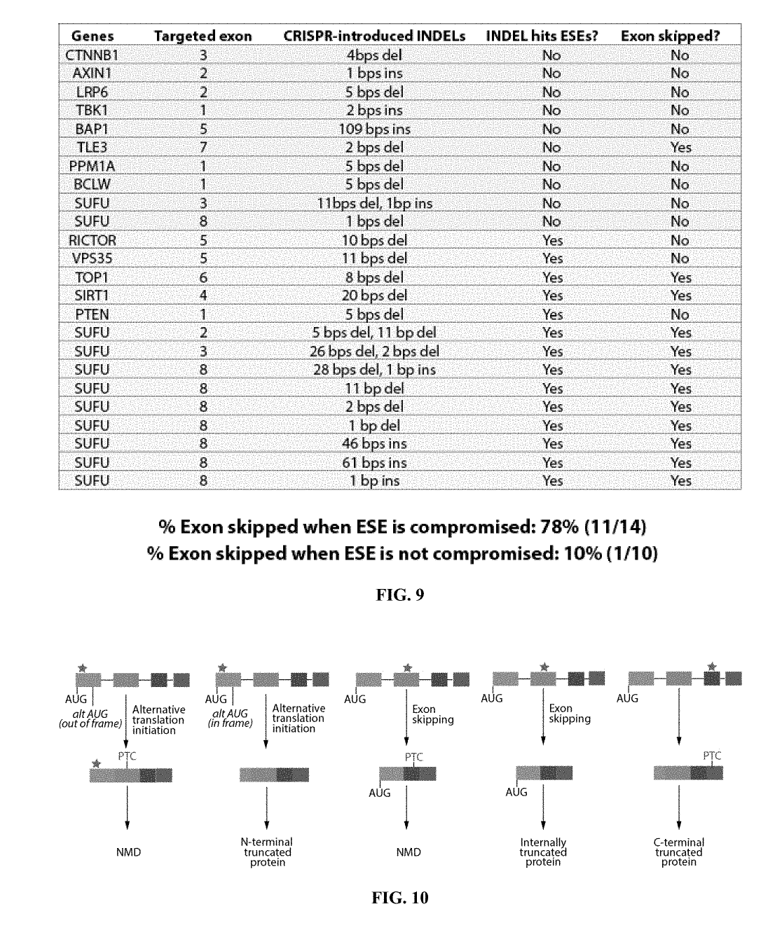

A flow chart depicting how CRISPR editing may affect protein production is pictured in FIG. 10. Indels from CRISPR editing in the first exon may result in alternative translation initiation and premature stop codons, therefore resulting in nonsense mediated decay. Alternatively, an indel may result in an alternate start codon being available, and result in the production of a protein with an N-terminal truncation (FIG. 10). Indels targeting exon splicing enhancers in subsequent exons may result in exon skipping and the introduction of premature stop codons and nonsense mediated decay or C-terminally truncated proteins, or internally truncated proteins which are missing the targeted exons (FIG. 10).

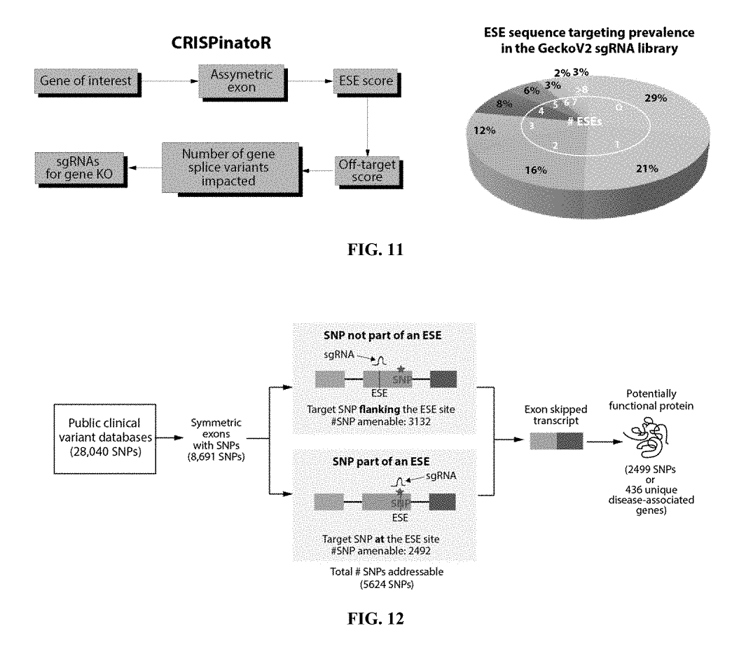

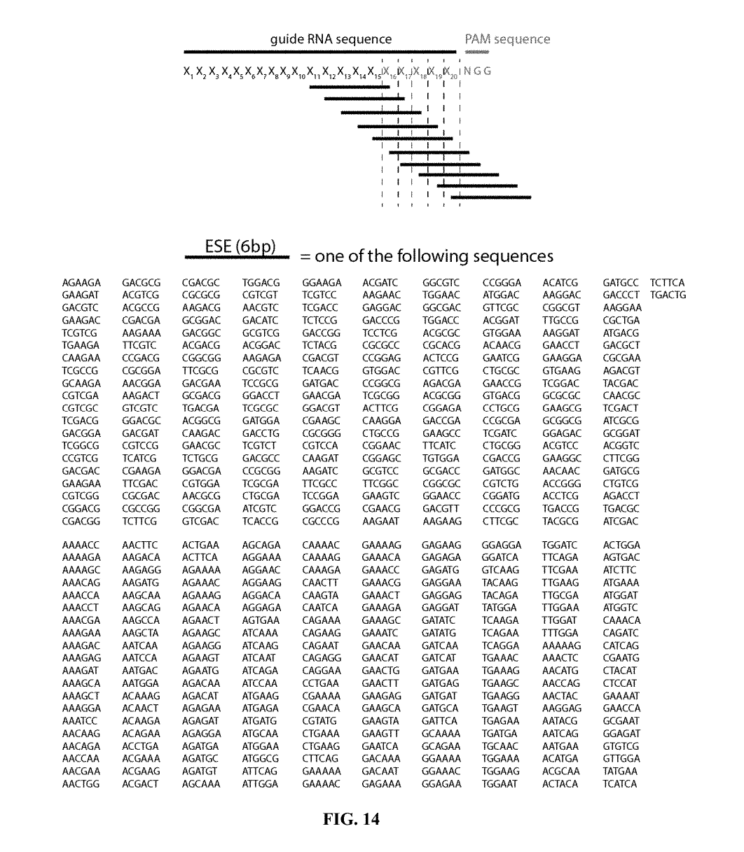

In order to specifically target guide RNAs to exploit predicted exon splicing enhancer elements found in exonic sequences, the CRISPinatoR web tool was developed (available on the world wide web at crispinator.com). CRISPinatoR sgRNA design follows the process shown in FIG. 11. Briefly, sgRNAs are targeted to a gene of interest, specifically to an asymmetric exon, and an ESE score is assigned based on the presence of putative ESEs. A list of putative ESEs is shown in Table 4 and the bottom of FIG. 14.

TABLE 4
6 bp Exon Splicing Enhancer Element Sequences

| | | | | | | | | | |
|---|---|---|---|---|---|---|---|---|---|
| AGAAGA | GACGCG | CGACGC | TGGACG | GGAAGA | ACGATC | GGCGTC | CCGGGA | ACATCG | GATGCC |
| GAAGAT | ACGTCG | CGCGCG | CGTCGT | TCGTCC | AAGAAC | TGGAAC | ATGGAC | AAGGAC | GACCCT |
| GACGTC | ACGCCG | AAGACG | AACGTC | TCGACC | GAGGAC | GGCGAC | GTTCGC | CGGCGT | AAGGAA |
| GAAGAC | CGACGA | GCGGAC | GACATC | TCTCCG | GACCCG | TGGACC | ACGGAT | TTGCCG | CGCTGA |
| TCGTCG | AAGAAA | GACGGC | GCGTCG | GACCGG | TCCTCG | ACGCGC | GTGGAA | AAGGAT | ATGACG |
| TGAAGA | TTCGTC | ACGACG | ACGGAC | TCTACG | CGCGCC | CGCACG | ACAACG | GAACCT | GACGCT |
| CAAGAA | CCGACG | CGGCGG | AAGAGA | CGACGT | CCGGAG | ACTCCG | GAATCG | GAAGGA | CGCGAA |
| TCGCCG | CGCGGA | TTCGCG | CGCGTC | TCAACG | GTGGAC | CGTTCG | CTGCGC | GTGAAG | AGACGT |
| GCAAGA | AACGGA | GACGAA | TCCGCG | GATGAC | CCGGCG | AGACGA | GAACCG | TCGGAC | TACGAC |
| CGTCGA | AAGACT | GCGACG | GGACCT | GAACGA | TCGCGG | ACGCGG | GTGACG | GCGCGC | CAACGC |
| CGTCGC | GTCGTC | TGACGA | TCGCGC | GGACGT | ACTTCG | CGGAGA | CCTGCG | GAAGCG | TCGACT |
| TCGACG | GGACGC | ACGGCG | GATGGA | CGAAGC | CAAGGA | GACCGA | CCGCGA | GCGGCG | ATCGCG |
| GACGGA | GACGAT | CAAGAC | GACCTG | CGCGGG | CTGCCG | GAAGCC | TCGATC | GGAGAC | GCGGAT |
| TCGGCG | CGTCCG | GAACGC | TCGTCT | CGTCCA | CGGAAC | TTCATC | CTGCGG | ACGTCC | ACGGTC |
| CCGTCG | TCATCG | TCTGCG | GACGCC | CAAGAT | CGGAGC | TGTGGA | CGACCG | GAAGGC | CTTCGG |
| GACGAC | CGAAGA | GGACGA | CCGCGG | AAGATC | GCGTCC | GCGACC | GATGGC | AACAAC | GATGCG |
| GAAGAA | TTCGAC | CGTGGA | TCGCGA | TTCGCC | TTCGGC | CGGCGC | CGTCTG | ACCGGG | CTGTCG |
| CGTCGG | CGCGAC | AACGCG | CTGCGA | TCCGGA | GAAGTC | GGAACC | CGGATG | ACCTCG | AGACCT |
| CGGACG | CGCCGG | CGGCGA | ATCGTC | GGACCG | CGAACG | GACGTT | CCCGCG | TGACCG | TGACGC |
| CGACGG | TCTTCG | GTCGAC | TCACCG | CGCCCG | AAGAAT | AAGAAG | CTTCGC | TACGCG | ATCGAC |
| AAAACC | AACTTC | ACTGAA | AGCAGA | CAAAAC | GAAAAG | GAGAAG | GGAGGA | TGGATC | ACTGGA |
| AAAAGA | AAGACA | ACTTCA | AGGAAA | CAAAAG | GAAACA | GAGAGA | GGATCA | TTCAGA | AGTGAC |
| AAAAGC | AAGAGG | AGAAAA | AGGAAC | CAAAGA | GAAACC | GAGATG | GTCAAG | TTCGAA | ATCTTC |
| AAACAG | AAGATG | AGAAAC | AGGAAG | CAACTT | GAAACG | GAGGAA | TACAAG | TTGAAG | ATGAAA |
| AAACCA | AAGCAA | AGAAAG | AGGACA | CAAGTA | GAAACT | GAGGAG | TACAGA | TTGCGA | ATGGAT |
| AAACCT | AAGCAG | AGAACA | AGGAGA | CAATCA | GAAAGA | GAGGAT | TATGGA | TTGGAA | ATGGTC |
| AAACGA | AAGCCA | AGAACT | AGTGAA | CAGAAA | GAAAGC | GATATC | TCAAGA | TTGGAT | CAAACA |
| AAAGAA | AAGCTA | AGAAGC | ATCAAA | CAGAAG | GAAATC | GATATG | TCAGAA | TTTGGA | CAGATC |
| AAAGAC | AATCAA | AGAAGG | ATCAAG | CAGAAT | GAACAA | GATCAA | TCAGGA | AAAAAG | CATCAG |
| AAAGAG | AATCCA | AGAAGT | ATCAAT | CAGAGG | GAACAT | GATCAT | TGAAAC | AAACTC | CGAATG |
| AAAGAT | AATGAC | AGAATG | ATCAGA | CAGGAA | GAACTG | GATGAA | TGAAAG | AACATG | CTACAT |
| AAAGCA | AATGGA | AGACAA | ATCCAA | CCTGAA | GAACTT | GATGAG | TGAAGC | AACCAG | CTCCAT |
| AAAGCT | ACAAAG | AGACAT | ATGAAG | CGAAAA | GAAGAG | GATGAT | TGAAGG | AACTAC | GAAAAT |
| AAAGGA | ACAACT | AGAGAA | ATGAGA | CGAACA | GAAGCA | GATGCA | TGAAGT | AAGGAG | GAACCA |
| AAATCC | ACAAGA | AGAGAT | ATGATG | CGTATG | GAAGTA | GATTCA | TGAGAA | AATACG | GCGAAT |
| AACAAG | ACAGAA | AGAGGA | ATGCAA | CTGAAA | GAAGTT | GCAAAA | TGATGA | AATCAG | GGAGAT |
| AACAGA | ACCTGA | AGATGA | ATGGAA | CTGAAG | GAATCA | GCAGAA | TGCAAC | AATGAA | GTGTCG |
| AACCAA | ACGAAA | AGATGC | ATGGCG | CTTCAG | GACAAA | GGAAAA | TGGAAA | ACATGA | GTTGGA |
| AACGAA | ACGAAG | AGATGT | ATTCAG | GAAAAA | GACAAT | GGAAAC | TGGAAG | ACGCAA | TATGAA |
| AACTGG | ACGACT | AGCAAA | ATTGGA | GAAAAC | GAGAAA | GGAGAA | TGGAAT | ACTACA | TCATCA |
| TCTTCA | TGACTG | | | | | | | | |

The off-targeting potential and the number of splice variants potentially impacted by any given sgRNA is also incorporated into an overall scoring system that rank orders a list of sgRNAs useful for gene elimination. Application of the CRISPinatoR tool to the GeckoV2 sgRNA library shows the prevalence of ESEs in the sgRNA library (FIG. 11, right), and more than 70% of sgRNAs found in the library are anticipated to impact one or more ESEs, potentially making some sgRNAs more effective at promoting gene elimination by targeting asymmetric exons, and others more likely to induce foreign protein expression by targeting symmetric exons.

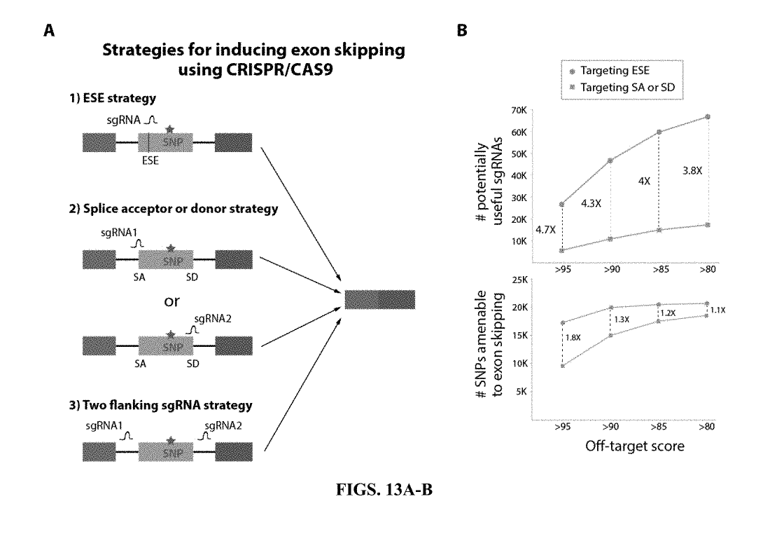

The CRISPinatoR web tool may also be used to design sgRNAs for gene rescue, by targeting ESEs using the CRISPR-skipper workflow (FIG. 12). SNPs associated with diseases (OMIM and SwissVar databases) found in symmetric exons are identified, and predicted ESEs that include the SNP or flank the SNP amenable to CRISPR attack are then used to identify sgRNAs with exon skipping potential and low off-targeting scores. Predicted proteins following exon skipping that do not have compromised annotated domain features are identified as viable rescue genes. This approach is compared to other strategies in which splice acceptor or donors are edited, or by using a strategy incorporating two sgRNAs (FIG. 13A). Analysis of these techniques is shown in FIG. 13B. Briefly, by targeting ESEs rather than splice acceptor or splice donors, there are significantly more potentially useful sgRNAs for use by targeting ESEs rather than targeting splice donors and acceptors (FIG. 13B). For example, at an off target score of >95, there are 4.7-fold more sgRNAs for targeting ESEs than there are for targeting splice donors or acceptors. Further, there are nearly two-fold more SNPs amenable to exon skipping when targeting ESEs compared to splice acceptors and donors.

Design of guide RNA sequences for CRISPR mediated gene editing by targeting ESEs is shown in FIG. 14. The guide RNA sequence follows the canonical pattern of X₂₀NGG for CRISPR/Cas9 systems, wherein the NGG is the protospacer adjacent motif. However the 6 bp ESE sequence is positioned in the guide RNAs such that at least one nucleotide of the ESE is positioned between the 16^{th} and the 20^{th} nucleotide (FIG. 14). A list of the putative ESEs is shown in Table 4 and at the bottom of FIG. 14. Guide RNAs can be designed to disrupt these ESEs as a part of a gene deletion campaign, or in order to rescue genes with disease associated SNPs.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Barnes et al., J. Biol. Chem., 272(17):11510-7, 1997
Benvenisty and Neshif, Proc. Natl. Acad. Sci. USA, 83:9551-9555, 1986.
Bhaysar et al., Genomics, 35(1):11-23, 1996.
Bikard et al., Nucleic Acids Res., 41(15): 7429-7437, 2013.
Caceres et al., Genome Biology, 14:R143, 2013.
Chen and Okayama, Mol Cell Biol., 7(8):2745-52, 1987.
Cho et al., Nat. Biotechnol., 31(3): 230-232, 2013.
Coffin, In: Virology, Fields et al. (Eds.), Raven Press, NY, 1437-1500, 1990.
Coupar et al., Gene, 68:1-10, 1988.
Donnelly et al., J. Gen. Virol., 82, 1027-1041, 2001.
Dubensky et al., Proc. Natl. Acad. Sci. USA, 81:7529-7533, 1984.
Ferkol et al., J Clin Invest., 92(5):2394-400, 1993.
Fraley et al., Proc Nat'l Acad. Sci. USA, 76:3348-3352, 1979.
Friedmann, Science, 244:1275-1281, 1989.
Fu et al., Nature Reviews Genetics 15: 689-701, 2014.
Ghosh and Bachhawat, Targeted Diagn Ther., 4:87-103, 1991.
Ghosh-Choudhury et al., EMBO J, 6:1733-1739, 1987.
Gomez-Foix et al., J. Biol. Chem., 267:25129-25134, 1992.
Graham and Prevec, In: Methods in Molecular Biology: Gene Transfer and Expression Protocol, Murray (Ed.), Humana Press, Clifton, N.J., 7:109-128, 1991.
Graham et al., J. Gen. Virol., 36:59-72, 1977.
Graham and van der Eb, Virology, 52:456-467, 1973.
Gopal et al., Mol. Cell Biol., 5:1188-1190, 1985.
Grunhaus and Horwitz, Seminar in Virology, 3:237-252, 1992.
Hermonat and Muzycska, Proc. Nat'l Acad. Sci. USA, 81:6466-6470, 1984.
Hersdorffer et al., DNA Cell Biol., 9:713-723, 1990
Herz and Gerard, Proc. Nat'l. Acad. Sci. USA, 90:2812-2816, 1993
Horwich et al., J. Virol., 64:642-650, 1990.
Hsu et al., Natl Biotechnol., 31:827-832, 2013
Hsu et al., Cell, 157: 1262-1278, 2014.
Jinek et al., Science, 337(6096):816-821, 2012.
Jones and Shenk, Cell, 13:181-188, 1978.
Kaneda et al., J. Biol. Chem., 264(21):121296-9, 1989.
Karlsson et al., EMBO 1, 5:2377-2385, 1986.
Kato et al., J. Biol. Chem., 266(6):3361-4, 1991.
Kelly et al., J. Cell Biol., 129(2):383-96, 1995.
Kimura et al., Dev. Growth Differ., 39(3):257-65, 1997.
Klein et al., Nature, 327:70-73, 1987.
LaPointe et al., Hypertension, 27(3):715-22, 1996
LaPointe et al., J. Biol. Chem., 263(19):9075-8, 1988.
Le Gal La Salle et al., Science, 259:988-990, 1993.
Levrero et al., Gene, 101:195-202, 1991.
Mali et al., Science, 339, 823-826, 2013a.
Mali et al., Nat Methods, 10, 957-963, 2013b.
Mali et al., Nat. Biotechnol., 31:833-838, 2013c
Mann et al., Cell, 33:153-159, 1983.
Markowitz et al., J. Virol., 62:1120-1124, 1988.
Moss et al., J. Gen. Physiol., 108(6):473-84, 1996.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-76, 1987.
Pennisi, Science, 341:833-836, 2013.
Perales et al., Eur J. Biochem., 226(2):255-66, 1994.
Racher et al., Biotech. Techniques, 9:169-174, 1995.
Ragot et al., Nature, 361:647-650, 1993.
Renan, Radiother. Oncol., 19:197-218, 1990.
Rich et al., Hum. Gene Ther., 4:461-476, 1993.
Ridgeway, In: Vectors: A survey of molecular cloning vectors and their uses, Stoneham:Butterworth, 467-492, 1988.
Ripe et al., Mol. Cell. Biol., 9:2224, 1989.
Rippe et al., Mol. Cell Biol., 10:689-695, 1990.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Temin, In: Gene Transfer, Kucherlapati (Ed.), NY, Plenum Press, 149-188, 1986.
Thiesen et al., J Virology, 62:614, 1988.
Tur-Kaspa et al., Mol. Cell Biol., 6:716-718, 1986.
Wagner et al., Proc Natl Acad Sci USA., 87(10):3655-9, 1990.
Wang et al., Cell, 153(4):910-918, 2013.
Wong et al., Gene, 10(2):87-94, 1980.
Wu and Wu, Biochem., 27:887-892, 1988.
Wu and Wu, J Biol Chem., 262(10):4429-32, 1987.
Yamashita et al., Nature, 408: 92-96, 2000.
Yamauchi-Takihara et al., Proc. Natl. Acad. Sci. USA, 86(10):3504-3508, 1989.
Yang et al., Proc. Nat'l Acad. Sci. USA, 87:9568-9572, 1990.
Zelenin et al., FEBS Lett., 280:94-96, 1991.
Zetsche et al., CeU 163: 1-13, 2015.
Ziober and Kramer, J Bio. Chem., 271(37):22915-22922, 1996.

1. A method for reducing the expression of a target gene in a cell, wherein the target gene comprises a putative exon splicing enhancer sequence, comprising contacting the cell with a gene editing construct and at least one polynucleotide sequence capable of hybridizing to a sequence comprising a putative exon splicing enhancer, or capable of hybridizing to a sequence adjacent to a putative exon splicing enhancer, within the target gene.

2. The method of claim 1, wherein the putative exon splicing enhancer sequence is selected from the sequences from Table 4.

3. The method of claim 1, wherein said polynucleotide sequence is a vector.

4. The method of claim 3, wherein the vector encodes a guide RNA.

5-6. (canceled)

7. The method of claim 4, wherein the guide RNA sequence comprises a sequence capable of hybridizing to a sequence adjacent to a putative exon splicing enhancer.

8. The method of claim 4, wherein the guide RNA sequence can hybridize to a sequence comprising at least 1, 2, 3, 4, or 5 nucleotides of putative exon splicing enhancer sequence, selected from Table 4, present in the target gene.

9. The method of claim 4, wherein the guide RNA sequence comprises at least one nucleotide of a protospacer adjacent motif (PAM) sequence.

10-11. (canceled)

12. The method of claim 1, wherein the vector is an expression vector.

13. (canceled)

14. The method of claim 1, wherein the polynucleotide sequence is an oligonucleotide.

15. The method of claim 1, wherein the oligonucleotide is a guide RNA.

16-17. (canceled)

18. The method of claim 15, wherein the guide RNA sequence comprises a sequence capable of hybridizing to a sequence adjacent to a putative exon splicing enhancer.

19. (canceled)

20. The method of claim 15, wherein the guide RNA sequence can hybridize to a sequence comprising at least 1, 2, 3, 4, or 5 nucleotides of putative exon splicing enhancer sequence, selected from Table 4, present in the target gene.

21. The method of claim 15, wherein the guide RNA sequence comprises at least one nucleotide of a PAM motif.

22-23. (canceled)

24. The method of claim 1, wherein the gene editing construct comprises a CRISPR-Cas9, CRISPR-Cas12a, or CRISPR-Cpf1 gene editing construct.

25. The method of claim 1, wherein the gene editing construct and at least one polynucleotide sequence are co-administered.

26. The method of claim 1, wherein the gene editing construct is administered prior to the administration of the at least one polynucleotide sequence.

27. The method of claim 1, wherein the gene editing construct is administered subsequent to the administration of the at least one polynucleotide sequence.

28. The method of claim 1, wherein the method further comprises disrupting the putative exon splicing enhancer by inserting or deleting a nucleotide within the sequence during gene editing.

29. A method for skipping expression of a target exon of a gene in a cell, wherein the target exon comprises a putative exon splicing enhancer sequence or is adjacent to a putative exon splicing enhancer sequence, comprising contacting the cell with a gene editing construct and at least one polynucleotide sequence capable of hybridizing to a sequence comprising said putative exon splicing enhancer or hybridizing adjacent to said putative exon splicing enhancer.

30-55. (canceled)

56. A method for treating a subject having a disease caused by a single nucleotide polymorphism in an exon, wherein the exon comprises or is adjacent to an exon splicing enhancer, comprising administering to the subject a therapeutically effective amount of a gene editing construct and at least one polynucleotide sequence capable of hybridizing to a sequence comprising or adjacent to said exon splicing enhancer.
